(19) United States
(12) Patent Application Publication
(10) Pub. No.: US 2004/0063761 A1
(43) Pub. Date: Apr. 1, 2004

(54) 2-(biarylalkyl)amino-3-(fluoroalkanoylamino)pyridine derivatives

(76) Inventors: Scott D. Kuduk, Harleysville, PA (US); Mark G. Bock, Hatfield, PA (US); Dong-Mei Feng, Blue Bell, PA (US); Jenny Miu-Chun Wai, Harleysville, PA (US)

Correspondence Address: MERCK AND CO INC, P O BOX 2000, RAHWAY, NJ, 070650907

(21) Appl. No.: 10/634,966

(22) Filed: Aug. 5, 2003

Related U.S. Application Data
(60) Provisional application No. 60/401,454, filed on Aug. 6, 2002.

(51) Int. Cl. A61K 31/444; C 07D 4 1/02; C07D 213/54

(52) U.S. Cl. 514/332; 514/351; 546/264; 546/307

(57) ABSTRACT

Compounds disclosed herein are bradykinin B1 antagonist compounds useful in the treatment or prevention of symptoms such as pain and inflammation associated with the bradykinin B1 pathway.

### BACKGROUND OF THE INVENTION

This invention is directed to 2,3-diaminopyridine derivatives. In particular, this invention is directed to 2,3-diaminopyridine derivatives that are bradykinin antagonists or inverse agonists.

Bradykinin (“BK”) is a kinin which plays an important role in the pathophysiological processes accompanying acute and chronic pain and inflammation. Bradykinin (BK), like other kinins, is an autacoid peptide produced by the catalytic action of kallikrein enzymes on plasma and tissue precursors termed kininogens. The biological actions of BK are mediated by at least two major G-protein-coupled BK receptors termed B1 and B2. It is generally believed that B2 receptors, but not B1 receptors, are expressed in normal tissues and that inflammation, tissue damage or bacterial infection can rapidly induce B1 receptor expression. This makes the B1 receptor a particularly attractive drug target. The putative role of kinins, and specifically BK, in the management of pain and inflammation has provided the impetus for developing potent and selective BK antagonists. In recent years, this effort has been heightened with the expectation that useful therapeutic agents with analgesic and anti-inflammatory properties would provide relief from maladies mediated through a BK receptor pathway (see e.g., M. G. Bock and J. Longmore, Current Opinion in Chem. Biol., 4:401-406(2000)). Accordingly, there is a need for novel compounds that are effective in blocking or reversing activation of bradykinin receptors. Such compounds would be useful in the management of pain and inflammation, as well as in the treatment or prevention of diseases and disorders mediated by bradykinin; further, such compounds are also useful as research tools (in vivo and in vitro).

U.S. Pat. No. 5,250,548 (Abbott) discloses angiotensin II receptor antagonists of the formula:

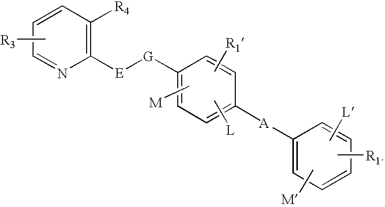

EP627433 (Eisai) discloses compounds of the formulae:

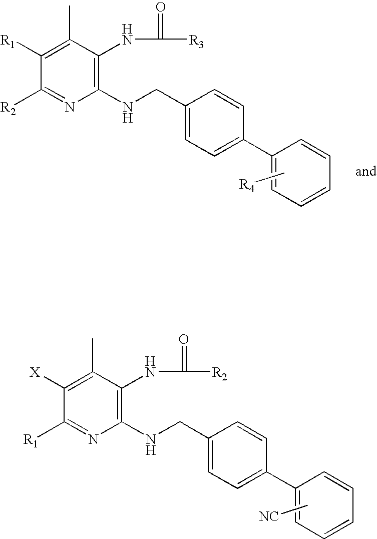

These compounds are intermediates in the process for the preparation of angiotensin II receptor antagonists.

EP470,543 (Karl Thomae) discloses the following generic formula as intermediates in the process for the preparation of angiotensin II receptor antagonists:

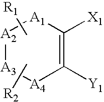

wherein one of X₁ and Y₁ is

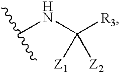

and the other is

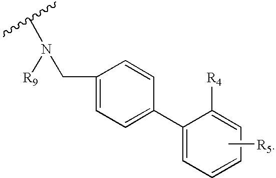

### SUMMARY OF THE INVENTION

The present invention provides N2, N3-disubstituted pyridine-2,3-diamine derivatives which are bradykinin antagonists or inverse agonists, pharmaceutical compositions containing such compounds, and methods of using them as therapeutic agents.

### DETAILED DESCRIPTION OF THE INVENTION

The present invention provides compounds of formula I:

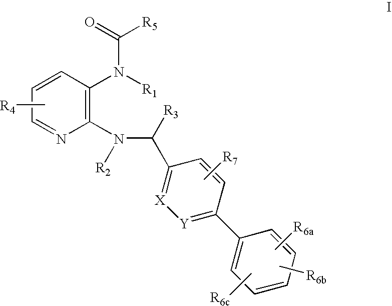

wherein

X and Y are each CH, or one is CH and the other is N;

R₁ and R₂ are independently selected from

(1) hydrogen and

(2) C₁₋₄ alkyl;

R₃ is selected from

(1) hydrogen, and

(2) C₁₋₄ alkyl optionally substituted with 1 to 4 groups selected from halogen, CO₂R^{a}, OR^{a}, COR^{a} and cyano;

R⁴ is selected from

(1) hydrogen,

(2) nitro,

(3) halogen,

(4) (CH₂)ₙOR^{a},

(5) (CH₂)ₙCO₂R^{a},

(6) (CH₂)ₙCN,

(7) (CH₂)ₙNR^{b}R^{c},

(8) (CH₂)ₙNHC(O)CH₂CN,

(9) CONR^{b}R^{c}, and

(10) C₁₋₄ alkyl;

R₅ is selected from

(1) C₃₋₆ cycloalkyl substituted with 1 or 2 fluoro,

(2) CHF₂,

(3) CH₂CF₃,

(4) CF₂CF₃, and

(5) CH₂CH₂CF₃;

R₆ₐ is selected from

(1) C₁₋₈ alkyl, optionally substituted with 1 to 5 groups independently selected from halogen, nitro, cyano, COR^{a}, SO₂R^{d}, CO₂R^{a}, NR^{b}R^{c}, NR^{b}C(O)R^{a}, NHSO₂R^{d}, OR^{a}, OC(O)R^{a}, CONR^{b}R^{c},

(2) C₃₋₈ cycloalkyl,

(3) C₂₋₈ alkenyl optionally substituted with CO₂R^{a};

(4) halogen,

(5) OCF₃,

(6) cyano,

(7) nitro,

(8) NR^{b}R^{c},

(9) NR^{b}C(O)R^{a},

(10) NR^{b}CO₂Ra^{′}, wherein Ra^{′} is a non-hydrogen group selected from R^{a},

(11) CO₂R^{a},

(12) COR^{a},

(13) C(O)NR^{b}R^{c},

(14) C(O)NHOR^{a},

(15) OR^{a},

(16) OC(O)R^{a},

(17) S(O)ₙR^{a′}, wherein R^{a′} is a non-hydrogen group selected from R^{a},

(18) SO₂NHR^{c},

(19) NHSO₂R^{d},

(20) C(═NOR^{a})NR^{b}R^{c},

(21) C(═NOR^{a})R^{a}, and

(22) substituted or unsubstituted heterocycle where the heterocycle is selected from oxadiazole, tetrazole, triazole, pyrazole, oxazole, isoxazole, thiazole, 4,5-dihydro-oxazole, 4,5-dihydro-1,2,4-oxadiazol-5-one, and wherein said substituent is 1 to 3 groups independently selected from C₁₋₄alkyl optionally substituted with 1 to 5 halogen atoms, OR^{a}, or OC(O)R^{a};

R_{6b} and R_{6c} are independently selected from

(1) hydrogen, and

(2) a group from R₆ₐ; with the proviso that not more than one of R₆ₐ, R_{6b}, and R_{6c} is a heterocycle;

R₇ is selected from

(1) hydrogen,

(2) cyano,

(3) nitro,

(4) halogen,

(5) OR^{a},

(6) CO₂R^{a},

(7) CONR^{b}R^{c}, and

(8) C₁₋₄ alkyl;

R^{a} is selected from

(1) hydrogen,

(2) C₁₋₄ alkyl,

(3) C₃₋₆ cycloalkyl,

(4) aryl, and

(5) aryl-C₁₋₄ alkyl;

R^{b}and R^{c} are independently selected from

(1) hydrogen,

(2) C₁₋₄ alkyl optionally substituted with OR^{a},

(3) C₃₋₆ cycloalkyl,

(4) aryl, and

(5) aryl-C₁₋₄ alkyl; or

R^{b}and R^{c} together with the nitrogen atom to which they are attached form a 5- or 6-membered ring optionally containing a heteroatom selected from NR^{a}, O and S;

R^{d} is selected from

(1) C₁₋₄ alkyl, optionally substituted with 1 to 3 halogen atoms,

(2) aryl,

(3) aryl-C₁₋₄ alkyl, and

(4) NR^{b}R^{c};

n is 0, 1 or 2

a pharmaceutically acceptable salt thereof.

Examples of R₁ and R₂ in formula I are hydrogen, methyl, ethyl, n-propyl, isopropyl, n-butyl, isobutyl, sec-butyl and tert-butyl.

Examples of R₃ include hydrogen, methyl, ethyl, n-propyl, isopropyl, n-butyl, isobutyl, sec-butyl and tert-butyl, difluoromethyl, trifluoromethyl, hydroxymethyl, 2-hydroxyethyl, 2-methoxyethyl, 3-ethoxypropyl, 4-chlorobutyl, cyanomethyl, carboxymethyl, ethoxycarbonylmethyl, and the like.

Examples of R₄ include hydrogen, nitro, chloro, fluoro, bromo, iodo, hydroxy, methoxy, ethoxy, isopropoxy, butoxy, hydroxymethyl, 2-hydroxyethyl, carboxy, carboxymethyl, methoxycarbonylmethyl, t-butoxycarbonylmethyl, cyano, cyanomethyl, 2-cyanoethyl, amino, dimethylaminomethyl, 2-(methylamino)ethyl, carbamoyl, carbamoylmethyl, 2-(N,N-dimethylcarbamoyl)ethyl, 2-cyanoacetamido, and the like.

Examples of R₆ₐ include methyl, ethyl, propyl, isobutyl, pentyl, 2-ethylbutyl, 3-ethylhexyl, heptyl, trifluoromethyl, difluoromethyl, 2-chloroethyl, cyano-methyl, 1-hydroxyethyl, 2-(methoxy)ethyl, 3-(propoxy)propyl, acetylmethyl, formyl-methyl, 2-cyanoethyl, 3-hydroxypropyl, hydroxymethyl, aminomethyl, methylamino-methyl, 2-(methylamino)ethyl, carbamoylmethyl, 2-(N,N-dimethylcarbamoyl)ethyl, formylaminomethyl, acetylaminomethyl, formyloxymethyl, 2-(methoxycarbonyl)-ethyl, methanesulfonamidomethyl, cyclopropanoylaminomethyl, ethanesulfonamido-methyl, cyclopropyl, cyclobutyl, cyclopentyl, cyclohexyl, cycloheptyl, cyclooctyl, vinyl, allyl, 4-butenyl, chloro, fluoro, bromo, iodo, cyano, nitro, amino, methylamino, dimethylamino, methylethylamino, formamido, acetamido, methyl carbamate, ethyl carbamate, methyl carboxylate, ethyl carboxylate, propyl carboxylate, t-butyl carboxylate, cyclopentyl carboxylate, methyl acrylate, formyl, acetyl, propionyl, carbamoyl, N-methylcarbamoyl, N,N-dimethylcarbamoyl, N-ethyl-N-methyl-carbamoyl, N-(methoxy)carbamoyl, N-(2-hydroxyethyl)carbamoyl, N-(1,2-dihydroxy)ethylcarbamoyl, N-(2-hydroxy)propylcarbamoyl, carboxarnide oxime, methoxy, ethoxy, propoxy, isopropoxy, trifluoromethoxy, acetyloxy, 1-(hydroxyimino)ethyl, 1-(methoxyimino)ethyl, methylthio, methylsulfinyl, methyl-sulfonyl, sulfonamide, N-methylsulfonamide, N-(t-butyl)sulfonamide, N,N-dimethyl-sulfonamide, N,N-dimethylsulfamoylamino, tetrazolyl, 1- and 2-methyltetrazol-5-yl, 3-methyl-1,2,4-oxadiazolyl, 5-methyl-1,2,4-oxadiazolyl, 5-ethyl-1,2,4-oxadiazolyl, 5-hydroxymethyl-1,2,4-oxadiazolyl, 3-acetoxymethyl-1,2,4-oxadiazolyl, 5-fluoro-methyl-1,2,4-oxadiazolyl, 1,3,4-oxadiazol-2-yl, 2-oxazolyl, 4,5-dihydro-2-oxazolyl, 5-methyl-4,5-dihydro-2-oxazolyl, 4-methyl-4,5-dihydro-2-oxazolyl, 4,4-dimethyl-4,5-dihydro-2-oxazolyl, 4-methyl-2-thiazolyl, 5-methyl-1,2,4-triazol-3-yl, 3-methyl-1,2,4-triazol-5-yl, and the like.

Examples of R_{6b} and R_{6c} include hydrogen and those groups mentioned above for R₆ₐ.

Examples of R₇ include hydrogen, cyano, bromo, chloro, fluoro, iodo, nitro, methoxy, ethoxy, propoxy, t-butoxy, methyl carboxylate, ethyl carboxylate, t-butyl carboxylate, carboxamide, methylcarboxamide, dimethylcarboxamide, ethylmethylcarboxamide, methyl, ethyl, propyl, isopropyl, t-butyl, and the like.

In one subset of compounds of formula I, R₁ and R₂ are each hydrogen.

In another subset of compounds of formula I, R₃ is hydrogen.

In another subset of compounds of formula I, R₃ is C₁₋₄ alkyl. In one embodiment thereof, R₃ is methyl.

In another subset of compounds of formula I, R₄ is H or a 4-substituent selected from C₁₋₄ alkyl, (CH₂)ₙNHC(O)CH₂CN, (CH₂)ₙCO₂R^{a}, (CH₂)ₙCN, (CH₂)ₙNR^{b}R^{c}, and halogen. In one embodiment thereof R₄ is H or a 4-substituent selected from C₁₋₄ alkyl and halogen. In a second embodiment, R₄ is hydrogen, 4-chloro or 4-methyl.

In another subset of compounds of formula I, R₅ is CH₂CF₃.

In another subset of compounds of formula I, X and Y are both CH.

In another subset of compounds of formula I, one of X and Y is CH and the other is N.

In another subset of compounds of formula I, R₆ₐ is a 2-(or ortho-) substituent. In one embodiment thereof R₆ₐ is selected from CO₂R^{a}, CONR^{b}R^{c}, CONHOR^{a}, C₁₋₈ alkyl substituted with 1 to 5 halogen atoms, cyano, SO₂NHR^{c}, halogen, trifluoromethoxy, 1- and 2-methyltetrazol-5-yl, 1,2,4-oxadiazolyl, 3- and 5-methyl-1,2,4-oxadiazolyl, 3- and 5-trifluoromethyl-1,2,4-oxadiazolyl, 3-pyrazolyl, 1,2,4-triazolyl, 5-methyl-1,2,4-triazol-3-yl, and 3-methyl-1,2,4-triazol-5-yl. In a second embodiment, R₆ₐ is selected from methyl carboxylate, cyano, N-methyl-carboxamido and N-methylsulfonamido. In a third embodiment R₆ₐ is 1- or 2-methyltetrazolyl. In a fourth embodiment, R₆ₐ is 1,2,4-oxadiazolyl, 5(4H)-oxo-1,2,4-oxadiazolyl, 4-methyl-5(4H)-oxo-1,2,4-oxadiazolyl, or 3- or 5-methyl-1,2,4-oxadiazolyl. In a fifth embodiment, R₆ₐ is difluoromethyl, trifluoromethyl, trifluoromethoxy, N-methoxycarboxamide, Cl, F or Br. In a sixth embodiment, R₆ₐ is 1,2,4-triazolyl, isoxazolyl or pyrazolyl.

In another subset of compounds of formula I, R_{6b} is selected from hydrogen, C₁₋₈ alkyl optionally substituted with OH or 1 to 5 halogen atoms, C₂₋₆ alkenyl, NR^{b}R^{c}, OR^{a}, COR^{a}, CO₂R^{a}, NHCOR^{a}, NHSO₂R^{d} and halogen, and R_{6c} is hydrogen. In one embodiment thereof R_{6b} is hydrogen, halogen, CO₂R^{a}, COR^{a}, or C₁₋₄alkyl optionally substituted with hydroxy. In a second embodiment, R_{6b} is hydrogen, fluoro, chloro, or methyl.

In another subset of formula I are compounds represented by formula Ia:

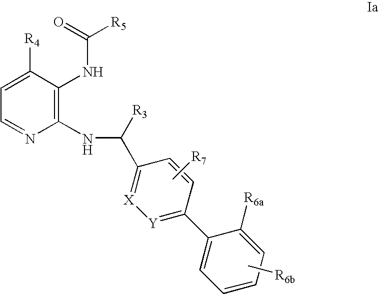

wherein R₃, R₄, R₅, R₆ₐ, R_{6b}, R₇, X and Y are as defined under formula I.

In one subset of formula Ia are compounds wherein at least one of R₃, R₄ and R_{6b} is non-hydrogen. In one embodiment thereof R₄ is C₁₋₄ alkyl or halogen. In a second embodiment thereof R₃ is C₁₋₄ alkyl. In a third embodiment therof R_{6b} is C₁₋₄ alkyl or halogen.

In another subset of formula Ia, at least two of R₃, R₄ and R_{6b} are non-hydrogen. In one embodiment thereof R₃ is C₁₋₄ alkyl and R_{6b} is C₁₋₄ alkyl or halogen. In a second embodiment thereof R₄ is C₁₋₄ alkyl or halogen and R_{6b} is C₁₋₄ alkyl or halogen. In a third embodiment thereof R₃ is C₁₋₄ alkyl and R₄ is C₁₋₄ alkyl or halogen. In a fourth embodiment thereof R₃ is C₁₋₄ alkyl, R₄ is C₁₋₄ alkyl or halogen and R_{6b} is C₁₋₄ alkyl or halogen.

In another subset of formula Ia are compounds wherein R₃ is hydrogen or methyl.

In another subset of formula Ia are compounds wherein R₄ is hydrogen, C₁₋₄ alkyl or halogen. In one embodiment thereof R₄ is hydrogen, methyl or chloro.

In another subset of formula Ia are compounds wherein R₅ is CH₂CF₃ or 2,2-difluorocyclopropyl. In one embodiment thereof R₅ is CH₂CF₃.

In another subset of formula Ia are compounds wherein R₆ₐ is selected from CO₂R^{a}, CONR^{b}R^{c}, CONHOR^{a}, C₁₋₈ alkyl substituted with 1 to 5 halogen atoms, cyano, SO₂NHR^{c}, halogen, trifluoromethoxy, 1- and 2-methyltetrazol-5-yl, 1,2,4-oxadiazolyl, 3- and 5-methyl-1,2,4-oxadiazolyl, 3- and 5-trifluoromethyl-1,2,4-oxadiazolyl, 3-pyrazolyl, 1,2,4-triazolyl, 5-methyl-1,2,4-triazol-3-yl, and 3-methyl-1,2,4-triazol-5-yl. In one embodiment thereof, R₆ₐ is selected from CO₂R^{a}, CONR^{b}R^{c}, SO₂NHR^{c}, and cyano. In a second embodiment R₆ₐ is selected from 1- or 2-methyltetrazolyl. In a third embodiment, R₆ₐ is 1,2,4-oxadiazolyl, 5(4H)-oxo-1,2,4-oxadiazolyl, 4-methyl-5(4H)-oxo-1,2,4-oxadiazolyl, or 3- or 5-methyl-1,2,4-oxadiazolyl. In a fourth embodiment, R₆ₐ is difluoromethyl, trifluoromethyl, trifluoromethoxy, N-methoxycarboxamide, Cl, F or Br. In a fifth embodiment, R₆ₐ is 1,2,4-triazolyl, isoxazolyl or pyrazolyl.

In another subset of formula Ia R_{6b} is hydrogen, or a 3-, 5- or 6-substituent selected from C₁₋₄alkyl and halogen. In one embodiment thereof, R_{6b} is hydrogen, methyl, chloro or fluoro.

In another subset of formula Ia X and Y are each CH and R₇ is hydrogen, halogen or C₁₋₄ alkyl; or one of X and Y is CH and the other is N, and R₇ is hydrogen.

In another subset of formula Ia, R₅ is CH₂CF₃, at least one of R₃, R₄ and R_{6b} is non-hydrogen, R₃ is hydrogen or C₁₋₄ alkyl, R₄ is H, C₁₋₄ alkyl, (CH₂)ₙNHC(O)CH₂CN, (CH₂)ₙCO₂R^{a}, (CH₂)ₙCN, (CH₂)ₙNR^{b}R^{c} or halogen, R₆ₐ is selected from CO₂R^{a}, CONR^{b}R^{c}, CONHOR^{a}, C₁₋₈ alkyl substituted with 1 to 5 halogen atoms, cyano, SO₂NHR^{c}, halogen, trifluoromethoxy, 1- and 2-methyl-tetrazol-5-yl, 1,2,4-oxadiazolyl, 3- and 5-methyl-1,2,4-oxadiazolyl, 3- and 5-trifluoro-methyl-1,2,4-oxadiazolyl, 3-pyrazolyl, 1,2,4-triazolyl, 5-methyl-1,2,4-triazol-3-yl, and 3-methyl-1,2,4-triazol-5-yl, R_{6b} is selected from hydrogen, C₁₋₈ alkyl optionally substituted with OH or 1 to 5 halogen atoms, C₂₋₆ alkenyl, NR^{b}R^{c}, OR^{a}, COR^{a}, CO₂R^{a}, NHCOR^{a}, NHSO₂R^{d} and halogen, and R_{6c} is hydrogen. In one embodiment thereof, R₄ is H, C₁₋₄ alkyl or halogen, R_{6b} is hydrogen, halogen, CO₂R^{a}, COR^{a}, or C₁₋₄alkyl optionally substituted with hydroxy; and within such embodiment, there is a group of compounds wherein R₆ₐ is selected from CO₂R^{a}, CONR^{b}R^{c}, SO₂NHR^{c}, and cyano; there is a second group of compounds wherein R₆ₐ is selected from 1- or 2-methyltetrazolyl; there is a third group of compounds wherein R₆ₐ is 1,2,4-oxadiazolyl, 5(4H)-oxo-1,2,4-oxadiazolyl, 4-methyl-5(4H)-oxo-1,2,4-oxadiazolyl, or 3- or 5-methyl-1,2,4-oxadiazolyl; there is a fourth group of compounds wherein R₆ₐ is difluoromethyl, trifluoromethyl, trifluoromethoxy, N-methoxycarboxamide, Cl, F or Br; and there is a fifth group of compounds wherein R₆ₐ is 1,2,4-triazolyl, isoxazolyl or pyrazolyl.

In another subset are compounds of formula I represented by formula Ib:

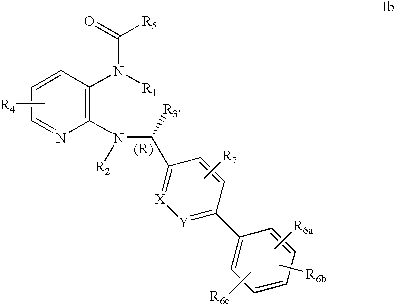

wherein all the variables are as defined under formula I, except R_{3′} is C₁₋₄ alkyl optionally substituted with 1 to 4 groups selected from halogen, CO₂R^{a}, OR^{a}, COR^{a} and cyano.

Unless otherwise stated, the following terms have the meanings indicated below:

“Alkyl” as well as other groups having the prefix “alk” such as, for example, alkoxy, alkanoyl, alkenyl, alkynyl and the like, means carbon chains which may be linear or branched or combinations thereof. Examples of alkyl groups include methyl, ethyl, propyl, isopropyl, butyl, sec- and tert-butyl, pentyl, hexyl, heptyl and the like.

“Alkenyl” means a linear or branched carbon chain containing at least one C═C bond. Examples of alkenyl include allyl, 2-butenyl, 3-butenyl, 1-methyl-2-propenyl, and the like.

“Aryl” means phenyl or naphthyl.

“Halogen” means fluorine, chlorine, bromine and iodine.

“Optionally substituted” is intended to include both substituted and unsubstituted. Thus, for example, optionally substituted aryl could represent a pentafluorophenyl or a phenyl ring.

### Optical Isomers—Diastereomers—Geometric Isomers—Tautomers

Compounds described herein may contain an asymmetric center and may thus exist as enantiomers. Where the compounds according to the invention possess two or more asymmetric centers, they may additionally exist as diastereomers. The present invention includes all such possible stereoisomers as substantially pure resolved enantiomers, racemic mixtures thereof, as well as mixtures of diastereomers. The above Formula I is shown without a definitive stereochemistry at certain positions. The present invention includes all stereoisomers of Formula I and pharmaceutically acceptable salts thereof. Diastereoisomeric pairs of enantiomers may be separated by, for example, fractional crystallization from a suitable solvent, and the pair of enantiomers thus obtained may be separated into individual stereoisomers by conventional means, for example by the use of an optically active acid or base as a resolving agent or on a chiral HPLC column. Further, any enantiomer or diastereomer of a compound of the general Formula I may be obtained by stereospecific synthesis using optically pure starting materials or reagents of known configuration.

Some of the compounds described herein contain olefinic double bonds, and unless specified otherwise, are meant to include both E and Z geometric isomers.

Some of the compounds described herein may exist with different points of attachment of hydrogen, referred to as tautomers. Such an example may be a ketone and its enol form known as keto-enol tautomers. The individual tautomers as well as mixture thereof are encompassed with compounds of Formula I.

### Salts

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Preferred are the ammonium, calcium, magnesium, potassium and sodium salts. Salts prepared from pharmaceutically acceptable organic non-toxic bases include salts of primary, secondary, and tertiary amines derived from both naturally occurring and synthetic sources. Pharmaceutically acceptable organic non-toxic bases from which salts can be formed include, for example, arginine, betaine, caffeine, choline, N,N′-dibenzyl-ethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, dicyclohexylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, its corresponding salt can be conveniently prepared from pharmaceutically acceptable non-toxic inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, hydrochloric, maleic, phosphoric, sulfuric, and tartaric acids.

### Prodrugs

The present invention includes within its scope prodrugs of the compounds of this invention. In general, such prodrugs will be functional derivatives of the compounds of this invention which are readily convertible in vivo into the required compound. Thus, in the methods of treatment of the present invention, the term “administering” shall encompass the treatment of the various conditions described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs,” ed. H. Bundgaard, Elsevier, 1985. Metabolites of these compounds include active species produced upon introduction of compounds of this invention into the biological milieu.

### Pharmaceutical Compositions

Another aspect of the present invention provides pharmaceutical compositions which comprises a compound of Formula I and a pharmaceutically acceptable carrier. The term “composition”, as in pharmaceutical composition, is intended to encompass a product comprising the active ingredient(s), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of Formula I, additional active ingredient(s), and pharmaceutically acceptable excipients.

The pharmaceutical compositions of the present invention comprise a compound represented by Formula I (or pharmaceutically acceptable salts thereof) as an active ingredient, a pharmaceutically acceptable carrier and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds represented by Formula I, or pharmaceutically acceptable salts thereof, of this invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compound represented by Formula I, or pharmaceutically acceptable salts thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound or a pharmaceutically acceptable salt of Formula I. The compounds of Formula I, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.1 mg to about 500 mg of the active ingredient and each cachet or capsule preferably containing from about 0.1 mg to about 500 mg of the active ingredient.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, utilizing a compound represented by Formula I of this invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in moulds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound described by Formula I, or pharmaceutically acceptable salts thereof, may also be prepared in powder or liquid concentrate form.

The following are examples of representative pharmaceutical dosage forms for the compounds of Formula I:

| Injectable Suspension (I.M.) | mg/mL |
|---|---|
| Compound of Formula I | 10 |
| Methylcellulose | 5.0 |
| Tween 80 | 0.5 |
| Benzyl alcohol | 9.0 |
| Benzalkonium chloride | 1.0 |

Water for injection to a total volume of 1 mL

| Tablet | mg/tablet |
|---|---|
| Compound of Formula I | 25 |
| Microcrystalline Cellulose | 415 |
| Povidone | 14.0 |
| Pregelatinized Starch | 43.5 |
| Magnesium Stearate | 2.5 |
| | 500 |

| Capsule | mg/capsule |
|---|---|
| Compound of Formula I | 25 |
| Lactose Powder | 573.5 |
| Magnesium Stearate | 1.5 |
| | 600 |

### Utilities

Compounds of this invention are antagonists or inverse agonists of bradykinin receptor, in particular the bradykinin B1 receptor, and as such are useful in the treatment and prevention of diseases and conditions mediated through the bradykinin receptor pathway such as pain and inflammation. The compounds would be effective in the treatment or prevention of pain including, for example, visceral pain (such as pancreatitis, interstitial cystitis, renal colic), neuropathic pain (such as postherpetic neuralgia, nerve injury, the “dynias”, e.g., vulvodynia, phantom limb pain, root avulsions, painful traumatic mononeuropathy, painful polyneuropathy), central pain syndromes (potentially caused by virtually any lesion at any level of the nervous system), and postsurgical pain syndromes (eg, postmastectomy syndrome, postthoracotomy syndrome, stump pain)), bone and joint pain (osteoarthritis), repetitive motion pain, dental pain, cancer pain, myofascial pain (muscular injury, fibromyalgia), perioperative pain (general surgery, gynecological), chronic pain, dysmennorhea, as well as pain associated with angina, and inflammatory pain of varied origins (e.g. osteoarthritis, rheumatoid arthritis, rheumatic disease, teno-synovitis and gout).

Further, the compounds of this invention can also be used to treat hyperreactive airways and to treat inflammatory events associated with airways disease e.g. asthma including allergic asthma (atopic or non-atopic) as well as exercise-induced bronchoconstriction, occupational asthma, viral- or bacterial exacerbation of asthma, other non-allergic asthmas and “wheezy-infant syndrome”. Compounds of the present invention may also be used to treat chronic obstructive pulmonary disease including emphysema, adult respiratory distress syndrome, bronchitis, pneumonia, allergic rhinitis (seasonal and perennial), and vasomotor rhinitis. They may also be effective against pneumoconiosis, including aluminosis, anthracosis, asbestosis, chalicosis, ptilosis, siderosis, silicosis, tabacosis and byssinosis.

Compounds of the present invention may also be used for the treatment of inflammatory bowel disease including Crohn's disease and ulcerative colitis, irritable bowel syndrome, pancreatitis, nephritis, cystitis (interstitial cystitis), uveitis, inflammatory skin disorders such as psoriasis and eczema, rheumatoid arthritis and edema resulting from trauma associated with burns, sprains or fracture, cerebral edema and angioedema. They may be used to treat diabetic vasculopathy, diabetic neuropathy, diabetic retinopathy, post capillary resistance or diabetic symptoms associated with insulitis (e.g. hyperglycemia, diuresis, proteinuria and increased nitrite and kallikrein urinary excretion). They may be used as smooth muscle relaxants for the treatment of spasm of the gastrointestinal tract or uterus. Additionally, they may be effective against liver disease, multiple sclerosis, cardiovascular disease, e.g. atherosclerosis, congestive heart failure, myocardial infarct; neurodegenerative diseases, eg. Parkinson's and Alzheimers disease, epilepsy, septic shock e.g. as anti-hypovolemic and/or anti-hypotensive agents, headache including cluster headache, migraine including prophylactic and acute use, closed head trauma, cancer, sepsis, gingivitis, osteoporosis, benign prostatic hyperplasia and hyperactive bladder. Animal models of these diseases and conditions are generally well known in the art, and may be suitable for evaluating compounds of the present invention for their potential utilities. Finally, compounds of the present invention are also useful as research tools (in vivo and in vitro).

The compounds of this invention are useful in the treatment of pain and inflammation by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

The compounds would be effective in the treatment or prevention of pain including, for example, bone and joint pain (osteoarthritis), repetitive motion pain, dental pain, cancer pain, myofascial pain (muscular injury, fibromyalgia), perioperative pain (general surgery, gynecological) and chronic pain by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

In particular, inflammatory pain such as, for example, inflammatory airways disease (chronic obstructive pulmonary disease) would be effectively treated by the compounds of this invention by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

Further, the compounds of this invention can additionally be used to treat asthma, inflammatory bowel disease, rhinitis, pancreatitis, cystitis (interstitial cystitis), uveitis, inflammatory skin disorders, rheumatoid arthritis and edema resulting from trauma associated with burns, sprains or fracture by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

They may be used subsequent to surgical intervention (e.g. as post-operative analgesics) and to treat inflammatory pain of varied origins (e.g. osteoarthritis, rheumatoid arthritis, rheumatic disease, teno-synovitis and gout) as well as for the treatment of pain associated with angina, menstruation or cancer by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

They may be used to treat diabetic vasculopathy, post capillary resistance or diabetic symptoms associated with insulitis (e.g. hyperglycemia, diuresis, proteinuria and increased nitrite and kallikrein urinary excretion) by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

They may be used to treat inflammatory skin disorders such as psoriasis and eczema by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

They may be used as smooth muscle relaxants for the treatment of spasm of the gastrointestinal tract or uterus or in the therapy of Crohn's disease, ulcerative colitis or pancreatitis by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

Such compounds may be used therapeutically to treat hyperreactive airways and to treat inflammatory events associated with airways disease e.g. asthma, and to control, restrict or reverse airways hyperreactivity in asthma by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

They may be used to treat intrinsic and extrinsic asthma including allergic asthma (atopic or non-atopic) as well as exercise-induced broncho-constriction, occupational asthma, viral or bacterial exacerbated asthma, other non-allergic asthmas and “wheezy-infant syndrome” by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

They may also be effective against pneumoconiosis, including aluminosis, anthracosis, asbestosis, chalicosis, ptilosis, siderosis, silicosis, tabacosis and byssinosis was well as adult respiratory distress syndrome, chronic obstructive pulmonary or airways disease, bronchitis, allergic rhinitis, and vasomotor rhinitis by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

Additionally, they may be effective against liver disease, multiple sclerosis, atherosclerosis, Alzheimer's disease, septic shock e.g. as anti-hypovolemic and/or anti-hypotensive agents, cerebral edema, headache including cluster headache, migraine including prophylactic and acute use, closed head trauma, irritable bowel syndrome and nephritis by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

### Combination Therapy

Compounds of Formula I may be used in combination with other drugs that are used in the treatment/prevention/suppression or amelioration of the diseases or conditions for which compounds of Formula I are useful. Such other drugs may be administered, by a route and in an amount commonly used therefor, contemporaneously or sequentially with a compound of Formula I. When a compound of Formula I is used contemporaneously with one or more other drugs, a pharmaceutical composition containing such other drugs in addition to the compound of Formula I is preferred. Accordingly, the pharmaceutical compositions of the present invention include those that also contain one or more other active ingredients, in addition to a compound of Formula I. Examples of other active ingredients that may be combined with a compound of Formula I, either administered separately or in the same pharmaceutical compositions, include, but are not limited to: (1) morphine and other opiate receptor agonists including propoxyphene (Darvon); (2) non-steroidal antiinflammatory drugs (NSAIDs) including COX-2 inhibitors such as propionic acid derivatives (alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (flufenamic acid, meclofenamic acid, mefenamic acid, niflumic acid and tolfenamic acid), biphenylcarboxylic acid derivatives (diflunisal and flufenisal), oxicams (isoxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone), and the coxibs (celecoxib, valecoxib, rofecoxib and etoricoxib); (3) corticosteroids such as betamethasone, budesonide, cortisone, dexamethasone, hydrocortisone, methylprednisolone, prednisolone, prednisone and triamcinolone; (4) histamine H1 receptor antagonists such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, cetirizine, desloratadine, fexofenadine and levocetirizine; (5) histamine H2 receptor antagonists such as cimetidine, famotidine and ranitidine; (6) proton pump inhibitors such as omeprazole, pantoprazole and esomeprazole; (7) leukotriene antagonists and 5-lipoxygenase inhibitors such as zafirlukast, montelukast, pranlukast and zileuton; (8) drugs used for angina, myocardial ischemia including nitrates such as nitroglycerin and isosorbide nitrates, beta blockers such as atenolol, metoprolol, propranolol, acebutolol, betaxolol, bisoprolol, carteolol, labetalol, nadolol, oxprenolol, penbutolol, pindolol, sotalol and timolol, and calcium channel blockers such as diltiazam, verapamil, nifedipine, bepridil, felodipine, flunarizine, isradipine, nicardipine and nimodipine; (9) incontinence medications such as antimuscarinics, e.g., tolterodine and oxybutinin); (10) gastrointestinal antispasmodics (such as atropine, scopolamine, dicyclomine, antimuscarinics, as well as diphenoxylate); skeletal muscle relaxants (cyclobenzaprine, carisoprodol, chlorphenesin, chlorzoxazone, metaxalone, methocarbamol, baclofen, dantrolene, diazepam, or orphenadrine); (11) gout medications such as allopurinol, probenicid and colchicine; (12) drugs for rheumatoid arthritis such as methotrexate, auranofin, aurothioglucose and gold sodium thiomalate; (13) drugs for osteoporosis such as alendronate and raloxifene; decongestants such as pseudoephedrine and phenylpropanolamine; (14) local anesthetics; (15) anti-herpes drugs such as acyclovir, valacyclovir and famcyclovir; and (15) anti-emetics such as ondansetron and granisetron.

### Biological Evaluation

Assessing the Affinity of Selected Compounds to Bind to the Bradykinin B1 or B2 Receptor

Radioligand binding assays are performed using membranes from CHO cells that stably express the human, rabbit, rat, or dog B1 receptors or CHO cells that express the human B2 receptor. For all receptor types, cells are harvested from culture flasks in PBS/1 mM EDTA and centrifuged at 1000×g for 10 minutes. The cell pellets are homogenized with a polytron in ice cold 20 mM HEPES, 1 mM EDTA, pH 7.4 (lysis buffer) and centrifuged at 20,000×g for 20 minutes. The membrane pellets are rehomogenized in lysis buffer, centrifuged again at 20,000×g and the final pellets are resuspended at 5 mg protein/ml in assay buffer (120 mM NaCl, 5 mM KCl, 20 mM HEPES, pH 7.4) supplemented with 1% BSA and frozen at −80° C.

On the day of assay, membranes are centrifuged at 14,000×g for 5 minutes and resuspended to the desired protein concentration in assay buffer containing 100 nM enaliprilat, 140 μg/mL bacitracin and 0.1% BSA. 3H-des-arg10, leu9 kallidin is the radioligand used for the human and rabbit B1 receptors, 3H-des-arg10 kallidin is used for the rat and dog B1 receptors, and 3H-bradykinin is used to label the human B2 receptor.

For all assays, compounds are diluted from DMSO stock solutions with 4 μL added to assay tubes for a final DMSO concentration of 2%. This is followed by the addition of 100 μL radioligand and 100 μL of the membrane suspension. Nonspecific binding for the B1 receptor binding assays is determined using 1 μM des-arg10 kallidin and nonspecific binding for the B2 receptor is determined with 1 μM bradykinin. Tubes are incubated at room temperature (22° C.) for 60 minutes followed by filtration using a Tomtec 96-well harvesting system. Radioactivity retained by the filter is counted using a Wallac Beta-plate scintillation counter.

The compounds of this invention have affinity for the B1 receptor in the above assay as demonstrated by results of less than 5 μM. It is advantageous that the assay results be less than 1 μM, even more advantageous for the results be less than 0.5 μM. It is further advantageous that compounds of this invention have affinity for the bradykinin B1 receptor over the bradykinin B2 receptor; more advantageously, the affinity for the B1 receptor is at least 10 fold, and preferably over 100 fold, over that for the B2 receptor.

Assay for Bradykinin B1 Antagonists

B1 agonist-induced calcium mobilization was monitored using a Fluorescence Imaging Plate Reader (FLIPR). CHO cells expressing the B1 receptor were plated in 96 or 384 well plates and allowed to incubate in Iscove's modified DMEM overnight. Wells were washed two times with a physiological buffered salt solution and then incubated with 4 uM Fluo-3 for one hour at 37° C. The plates were then washed two times with buffered salt solution and 100 uL of buffer was added to each well. Plates were placed in the FLIPR unit and allowed to equilibrate for two minutes. The test compound was then added in 50 ul volumes followed five minutes later by 50 ul of agonist (des-arg¹⁰ kallidin). Relative fluorescence peak heights in the absence and presence of antagonist were used to calculate the degree of inhibition of the B1 receptor agonist response by the test compound. Eight to ten concentrations of test compound were typically evaluated to construct an inhibition curve and determine IC50 values using a four-parameter nonlinear regression curve fitting routine.

Assay for Bradykinin Inverse Agonists

Inverse agonist activity at the human B1 receptor was evaluated using transiently transfected HEK293 cells. One day following transfection cell flasks were labeled overnight with 6 uCi/ml [³H]myo-inositol. On the day of assay, the media was removed and the attached cells were gently rinsed with 2×20 ml of phosphate-buffered saline. Assay buffer (HEPES buffered physiological salts, pH 7.4) was added and the cells were detached by tapping of the flask. The cells were centrifuged at 800×g for five minutes and resuspended at 1×10⁶ cells/ml in assay buffer supplemented with 10 mM lithium chloride. After 10 minutes at room temperature, one-half ml aliquots were distributed to tubes containing test compound or vehicle. After an additional 10 minutes the tubes were transferred to a 37° C. water bath for 30 minutes. The incubation was terminated by the addition of a 12% perchloric acid solution and the tubes were placed on ice for 30 minutes. The acid was then neutralized with KOH and the tubes centrifuged to pellet precipitated material. [³H]Inositol monophosphate formed was recovered by standard ion exchange chromatographic techniques and quantitated by liquid scintillation counting. Inverse agonist activity was determined by the degree to which a test compound reduced basal (cells incubated with vehicle) levels of [³H]inositol monophosphate accumulation.

Abbreviations Used

| | |
|---|---|
| AIBN | 2,2′-azobisisobutyronitrile |
| Bu | butyl |
| DMF | dimethylformamide |
| DMSO | Dimethyl dimethyl sulfoxide |
| EDC or EDCI | 1-(3-dimethylaminopropyl)3-ethylcarbodiimide HCl |
| ES (or ESI) - MS | electron spray ionization - mass spectroscopy |
| EtOAc | ethyl acetate |
| HBT or HOBt | 1-hydroxybenzotriazole hydrate |
| HPLC | high pressure liquid chromatography |
| Me | methyl |
| MeOH | methanol |
| NBS | N-bromosuccinimde |
| NMR | nuclear magnetic resonance |
| Ph | phenyl |
| rt | room temperature |
| TEA | triethylamine |
| Tf | triflate (trifluoromethanesulfonyl) |
| TFA | trifluoroacetic acid |
| THF | tetrahydrofuran |

The compounds of the present invention can be prepared according to the following reaction schemes and examples, or modifications thereof, using readily available starting materials, reagents, and conventional synthesis procedures. In these reactions, it is also possible to make use of variants which are themselves known to those of ordinary skill in this art, but are not mentioned in greater detail.

In Scheme 1, alkylation of a 2-amino-3-nitropyridine derivative (1a) with a bromomethyl biphenyl derivative (2a) in an appropriate aprotic solvent like N,N-dimethylformamide and in the presence of a suitable base like sodium hydride yields a 3-nitropyridine intermediate (3). The latter compound can be reduced catalytically with hydrogen or with a metal, like tin, to give an amino derivative (4) which is then reacted with a carboxylic acid or carboxylic acid equivalent to yield the title compound (I′).

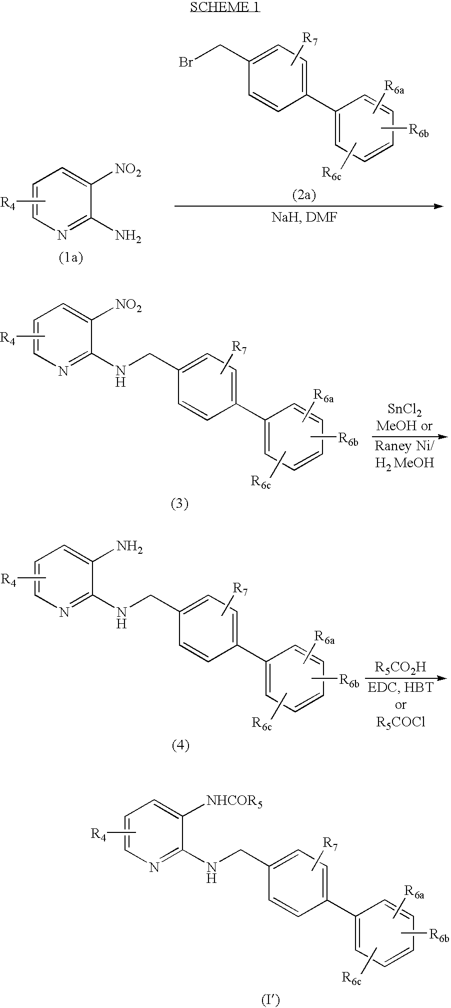

Alternatively, according to Scheme 2, the biphenyl moiety (7) is first assembled using a Suzuki reaction between an aromatic halide or triflate (5) and an aromatic boronic acid derivative (6) in the presence of triphenylphosphine and a metal catalyst like palladium acetate. The resultant biphenyl intermediate (7), also obtainable via an aryl zinc compound (8) as shown, is then reduced via a Raney Nickel reduction to afford the corresponding benzylic amine intermediate (2b). The latter compound is then reacted with a 2-chloro-3-nitropyridine derivative (1b) to afford the compound (3), which is reduced and then reacted with a carboxylic acid or carboxylic acid equivalent to yield the desired final product as illustrated in Scheme 1.

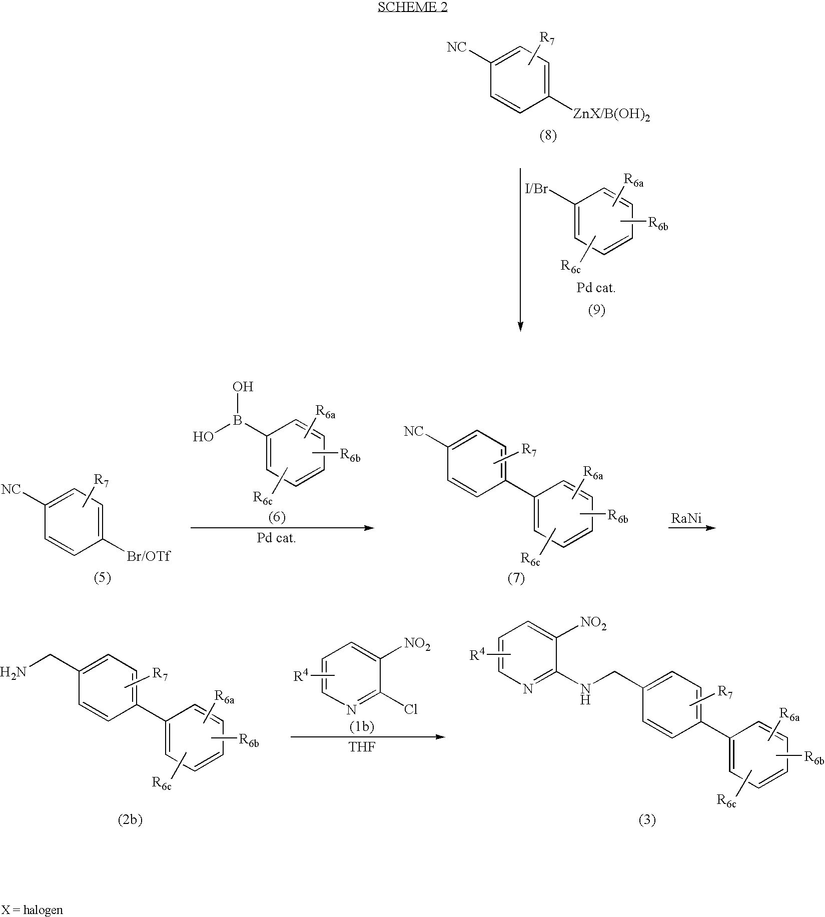

Alternatively, as illustrated in Scheme 3, the terminal phenyl group may be introduced on to intermediate (12a) via the formation of a pinacol boron ester in an aprotic solvent like dimethylsulfoxide. The former compound (12a) may be prepared from the appropriate benzylic amine with a 2-chloro-3-nitropyridine derivative (1b), or from a benzylic bromide with a 2-amino-3-nitropyridine, followed by reduction similar to Scheme 1. The boron ester (15) is coupled to an aryl halide derivative employing Suzuki reaction conditions to yield the penultimate product (4), which is converted to the title compound by reacting it with a carboxylic acid or carboxylic acid equivalent.

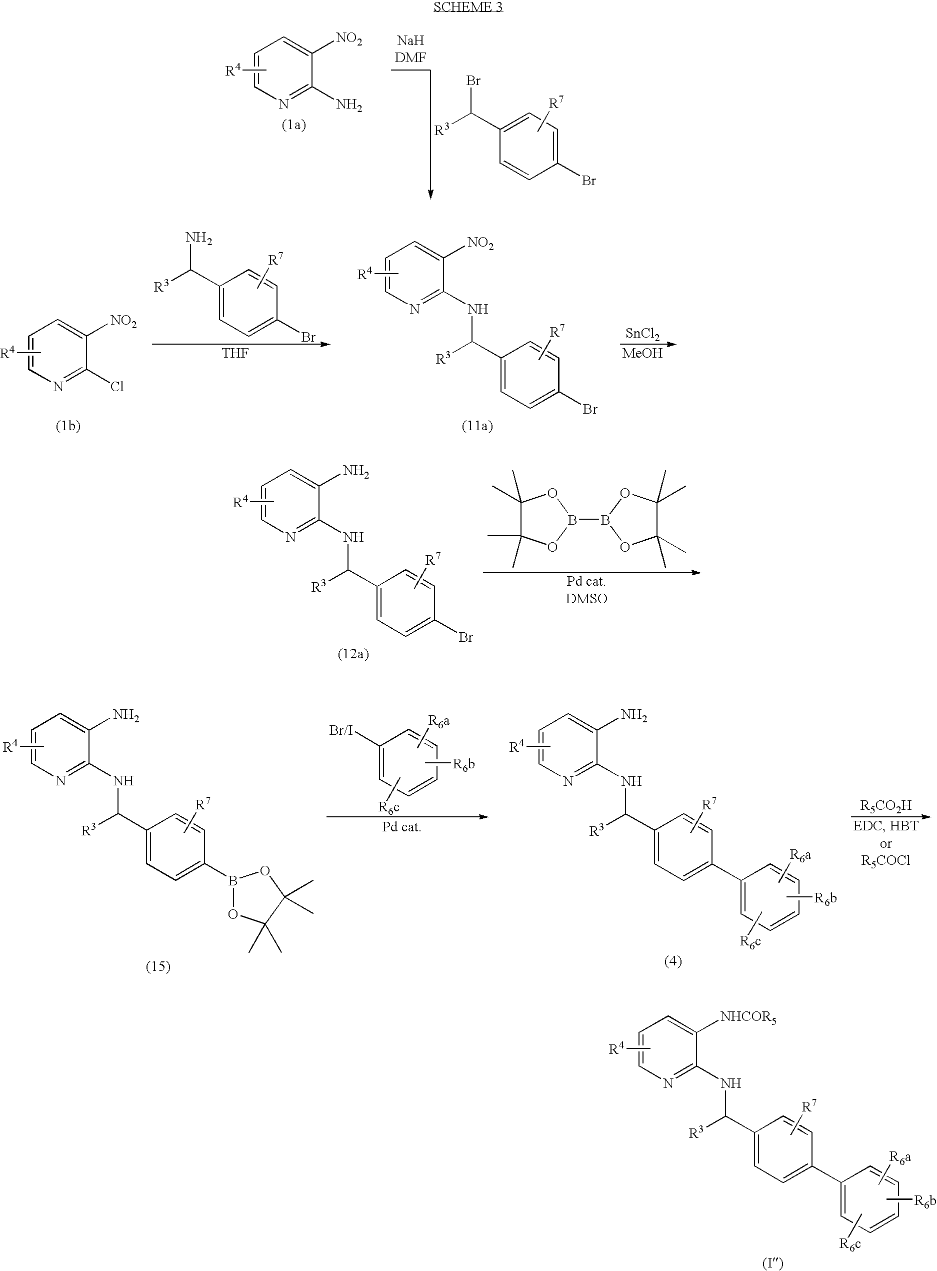

Another strategy can be employed to prepare compounds of the present invention according to Scheme 4. 2-Chloro-3-nitro-4-hydroxypyridine (1c) is heated with a 4-bromobenzylamine derivative (10b) in an appropriate solvent like n-butanol. The resulting adduct (11b) is converted to the 4-chloro derivative (11c) by the action of phosphorus oxychloride in an aprotic solvent like acetonitrile. Catalytic reduction of the nitro derivative (11c) with hydrogen or with a metal, like tin, to give an amino derivative (12b) is followed by the formation of a pinacol boron ester, coupling to an aryl halide derivative employing Suzuki reaction conditions, and acylation as described in Scheme 3 to provide the desired product (I′″).

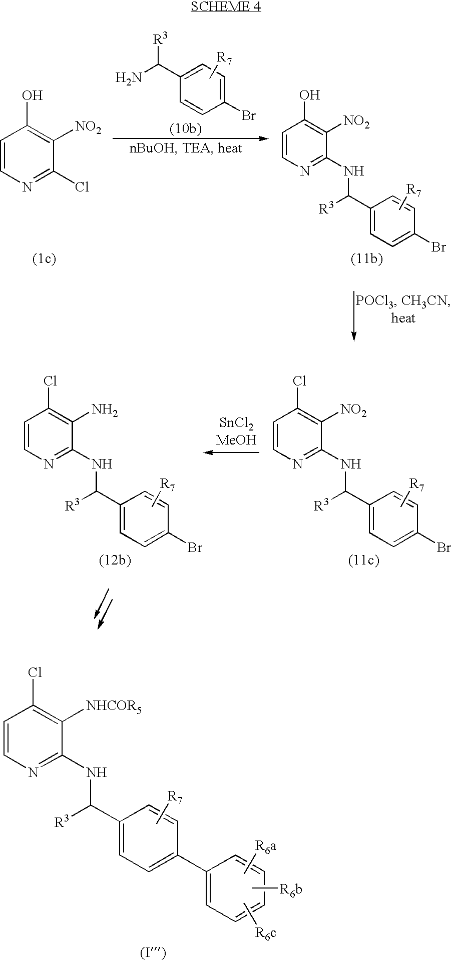

Additionally, according to Scheme 5, the biaryl moiety (7b) is first assembled using a palladium catalyzed coupling of (16) with an aryl zinc compound (17) as shown. The biaryl (7) is then elaborated at the benzylic position according to the three step sequence of halogenation, nucleophilic displacement of the halogen with azide, and reduction to the corresponding benzylic amine intermediate (2d). The latter compound is then reacted with a 2-chloro3-nitropyridine derivative, followed by reduction and then reaction with a carboxylic acid or carboxylic acid equivalent to yield the desired final product as illustrated in Scheme 1.

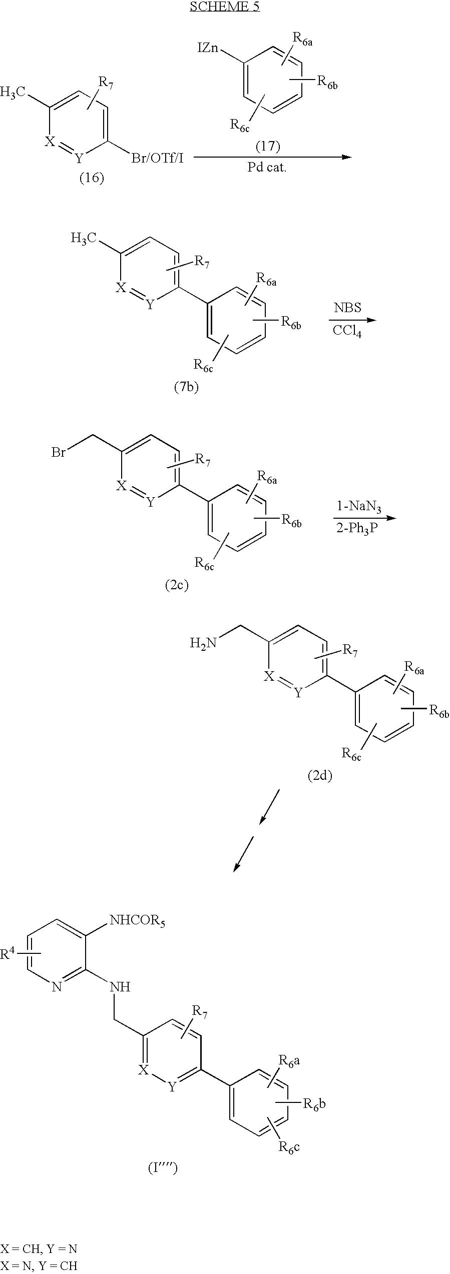

The following examples are provided to further illustrate the invention without, however, limiting the invention to the particulars of these examples.

### EXAMPLE 1 (METHOD A)

### Methyl 3,3′-Difluoro-4′-[({4-methyl-3-[(3,3,3-trifluoropropanoyl)amino]pyridin-2-yl}amino)methyl]-1,1′-biphenyl-2-carboxylate

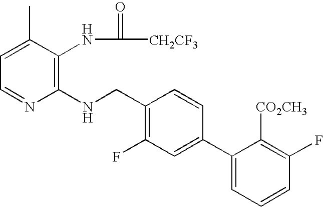

To a solution of 2-amino-4-methyl-3-nitropyridine (1.53 g, 10 mmol) in DMF (10 mL) at 0° C., sodium hydride (60% dispersion in mineral oil, 0.20 g, 5 mmol) was added, and stirred at 0° C. for 20 minutes. To the resulting mixture, 2-fluoro-4-bromobenzyl bromide (1.34 g, 5 mmol) was added, and stirred at 0° C. for another 10 minutes. The reaction was quenched by the addition of saturated ammonium chloride (10 mL), and partitioned between ethyl acetate and water. The organic extract was washed with brine, dried over MgSO₄, filtered and concentrated under vacuum to provide N-(4-bromo-2-fluorobenzyl)-4-methyl-3-nitropyridin-2-amine with a mass ion (ES+) of 340.1 for M+H⁺.

The above residue was suspended in methanol (20 mL), and tin(II) chloride dihydrate (4.51 g, 20 mmol) was added and heated in a sealed tube at 70° C. for 5 hours. The resulting solution was concentrated under vacuum. The residue was dissolved in ethyl acetate (100 mL), and 10% aq. sodium carbonate solution were added with vigorous stirring until pH=10. The white suspension was filtered through a pad of Celite, and the filtrate was partitioned between ethyl acetate and water. The organic extract was washed with brine, dried over MgSO₄, filtered and concentrated under vacuum. The residue was subjected to silica gel chromatography eluted with 40% ethyl acetate in hexanes to provide N-2-(4-bromo-2-fluorobenzyl)-4-methylpyridine-2,3-diamine (0.42 g, 1.35 mmol) as a yellow solid with a mass ion (ES+) of 310.2 for M+H⁺.

To a stirred solution of the above diamine (0.42 g, 1.35 mmol) in DMF (4 mL) in a sealed tube, bis(pinacolato)diboron (0.688 g, 2.71 mmol), dichloro[1,1′-bis(diphenylphosphino)ferrocene]palladium (II) dichloromethane adduct (0.0396 g, 0.05 mmol), and potassium acetate (0.399 g, 4.06 mmol) were added at room temperature. The resulting mixture was heated at 80° C. for 16 hours to provide N-2-[2-fluoro-4-(4,4,5,5-tetramethyl-1,3,2-dioxaborolan-2-yl)benzyl]-4-methylpyridine-2,3-diamine, and the solution was cooled to room temperature. To this solution, methyl 2-fluoro-6-iodobenzoate (0.756 g, 2.70 mmol), dichloro[1,1′-bis(diphenyl-phosphino)ferrocene]palladium (II) dichloromethane adduct (0.0395 g, 0.05 mmol), and sodium carbonate solution (2 M, 3.38 mL, 6.75 mmol) was added. The resulting mixture was heated at 80° C. for 16 hours. After cooling to room temperature, the mixture was partitioned between water and diethyl ether. The organic extract was washed with brine, dried over MgSO₄, filtered and concentrated under vacuum. The residue was subjected to silica gel chromatography eluted with 45% ethyl acetate in hexanes to provide methyl 4′-{[(3-amino-4-methylpyridin-2-yl)amino]methyl}-3,3′-difluoro-1,1′-biphenyl-2-carboxylate (0.50 g, 1.31 mmol) as a yellow oil with a mass ion (ES+) of 384.4 for M+H⁺.

To a solution of the above carboxylate (0.153 g, 0.40 mmol) in DMF (2 mL), 3,3,3-trifluoropropanoic acid (0.154 g, 1.20 mmol), 1-ethyl-(3-dimethyl-aminopropyl)carbodiimide hydrochloride (0.153 g, 0.80 mmol), 1-hydroxy-7-aza-benzotriazole (0.0544 g, 0.40 mmol) were added, and N,N-diisopropylethylamine was added until pH=8. The resulting solution was stirred at room temperature for 1 hour, and then partitioned between ethyl acetate and water. The organic extract was washed with brine, dried over MgSO₄, filtered and concentrated under vacuum. The residue was subjected to silica gel chromatography eluted with 40% ethyl acetate in hexanes. The appropriate fractions were concentrated under vacuum, and the residue was dissolved in 60 mL of 50% acetonitrile in hydrochloric acid (2 mL conc. hydrochloric acid in 4 L water). Lyophilization of the resulting solution afforded HCl salt of the title compound that gave a proton NMR spectrum consistent with theory and a mass ion (ES+) of 494.4 for M+H⁺: ¹H NMR (600 MHz, CD₃CN) δ 9.37 (br s, 1H), 7.76 (br s, 1H), 7.69 (d, J=6.6 Hz, 1H), 7.56 (m, J=5.8 Hz, 7.9 Hz, 1H), 7.50 (t, J=7.8 Hz, 1H), 7.28 (d, J=7.6 Hz, 1H), 7.25 (t, J=9.0 Hz, 1H), 7.15 (d, J=10 Hz, 1H), 7.12 (d, J=7.8 Hz, 1H), 6.80 (d, J=6.6 Hz, 1H), 4.96 (d, J=5.2 Hz, 2H), 3.67 (s, 3H), 3.60 (q, J=11 Hz, 2H), 2.25 (s, 3H).

### EXAMPLE 2 (METHOD B)

### N-[4-Chloro-2-({(1R)-1-[3′-fluoro-2′-(3-methyl-1,2,4-oxadiazol-5-yl)-1,1′-biphenyl-4-yl]ethyl}amino)pyridin-3-yl]-3,3,3-trifluoropropanamide

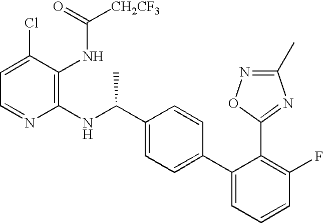

To a solution of 2-fluoro-6-iodobenzoic acid (10.0 g, 37.6 mmol) in 100 mL CH₂Cl₂ containing a catalytic amount of DMF, oxalyl chloride (6.20 g, 48.9 mmol) was added drop-wise. The solution was stirred at room temperature ˜30 minutes, and concentrated under vacuum. The residue was dissolved in 100 mL CH₂Cl₂ and saturated 3× with ammonia gas. The suspension was concentrated under vacuum to yield 2-fluoro-6-iodobenzamide as a white solid with a mass ion (ES+) of 266.4 for M+H⁺.

A solution of the above residue (11.5 g, 43.4 mmol) in N,N-dimethyl-acetamide dimethyl acetal (17.34 g, 130.2 mmol) was heated to 100° C. for 3 hours. The solution was concentrated under vacuum. Hydroxylamine hydrochloride (3.62 g, 52.1 mmol), 5N NaOH (10.4 mL, 52.1 mmol), 70% acetic acid (40 mL), and dioxane (40 mL) were combined and added to the above residue. The mixture was heated to 90° C. for 1 hour, cooled to room temperature, and concentrated under vacuum. The residue was diluted with EtOAc, washed with aqueous sodium bicarbonate and brine, dried over Na₂SO₄, filtered and concentrated under vacuum. The residue was subjected to column chromatography on silica gel eluted with 0-10% ethyl acetate in hexanes to afford 5-(2-fluoro-6-iodophenyl)-3-methyl-1,2,4-oxadiazole as a pale yellow solid with a mass ion (ES+) of 305.4 for M+H⁺.

A mixture of 4-chloro-N-2-{(1R)-1-[4-(4,4,5,5-tetramethyl-1,3,2-dioxaborolan-2-yl)phenyl]ethyl}pyridine-2,3-diamine (0.200 g, 0.535 mmol), 5-(2-fluoro-6-iodophenyl)-3-methyl-1,2,4-oxadiazole (0.179 g, 0.59 mmol), potassium carbonate (0.185 g, 1.34 mmol), tri-ortho-tolylphosphine (0.007 g, 0.02 mmol), and palladium acetate (3.0 mg, 0.01 mmol) in 4 mL of THF and 0.05 mL of water was heated in a sealed flask at 100° C. overnight. The mixture was then cooled and partitioned between ethyl acetate and water. The organic extract was washed with brine, dried over Na₂SO₄, filtered and concentrated under vacuum. The residue was subjected to silica gel chromatography eluted with 0-20% ethyl acetate and hexane to provide 4-chloro-N˜2˜-{(1R)-1-[3′-fluoro-2′-(3-methyl-1,2,4-oxadiazol-5-yl)-1-1,1′-biphenyl-4-yl]ethyl}pyridine-2,3-diamine with a mass ion (ES+) of 424.58 for M+H⁺(³⁵Cl).

To a solution of the above compound (0.050 mg, 0.12 mmol), 3,3,3-trifluoropropionic acid, (0.030 g, 0.24 mmol), 1-ethyl-(3-dimethylaminopropyl)-carbodiimide hydrochloride (0.045 g 0.24 mmol), 1-hydroxy-7-azabenzotriazole (0.023 g, 0.34 mmol) was added triethylamine (0.048 g, 0.47 mmol). The resulting solution was stirred at room temperature for 24 h, and partitioned between ethyl acetate and water. The organic extract was washed with brine, dried over Na₂SO₄, filtered and concentrated. The residue was subjected to silica gel chromatography eluted with 0-30% ethyl acetate in hexanes to provide the title compound that gave a proton NMR spectrum consistent with theory and a mass ion (ES+) of 534.6 for M+H⁺(³⁵Cl): ¹H NMR (300 MHz, MeOH-d₄) δ 7.81 (dd, J=5.6, 2.0 Hz, 1H), 7.75-7.67 (m, 1H), 7.39-7.30 (m, 4H), 7.12-7.09 (m, 2H), 6.68 (dd, J=5.4, 2.0 Hz, 1H), 5.25 (q, J=7.1 Hz, 1H), 3.32 (q, J=1.5 Hz, 2H), 2.33 (s, 3H), 1.53 (d, J=7.1 Hz, 3H).

### EXAMPLE 3 (METHOD C)

### Methyl 2-{6-[({4-Methyl-3-[(3,3,3-trifluoropropanoyl)amino]pyridin-2-yl}amino)methyl]pyridin-3-yl}benzoate

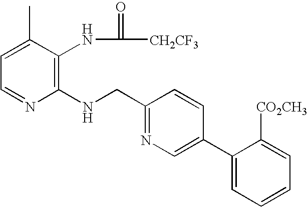

To a solution of 6-methylpyridin-3-yl trifluoromethanesulfonate (0.75 g, 3.11 mmol) and palladium tetrakistriphenylphosphine (0.36 g, 0.31 mmol) was added a solution of iodo[2-(methoxycarbonyl)phenyl]zinc (prepared from 2-iodo-methylbenzoate and Rieke Zinc) in THF via cannula. The mixture was heated to reflux for two hours, cooled, and partitioned between ethyl acetate and water. The organic extract was washed with brine, dried over MgSO₄, filtered and concentrated under vacuum. The residue was filtered through silica gel to afford methyl 2-(6-methylpyridin-3-yl)benzoate. A solution of the above compound (1.07 g, 4.71 mmol), N-bromosuccinimide (0.828 g, 4.71 mmol), and 2,2′-azobisisobutyronitrile (0.232 g, 1.41 mmol) in 20 mL carbon tetrachloride was heated to 90° C. for 5 hours. The resulting solution was cooled to room temperature and washed with water and brine, dried over Na₂SO₄, filtered and concentrated. The residue was subjected to silica gel chromatography eluted with 10-20% ethyl acetate in hexanes to provide methyl 2-[6-(bromomethyl)pyridin-3-yl]benzoate that gave a proton NMR spectrum consistent with theory and a mass ion (ES+) of 306.1 for M+H⁺ (Br⁷⁹).

A solution of methyl 2-[6-(bromomethyl)pyridin-3-yl]benzoate (0.568 g, 1.86 mmol) and sodium azide (0.245 g, 3.71 mmol) in 10 mL DMF was heated to 45° C. for 2 hours. The resulting solution was cooled to room temperature and partitioned between ethyl acetate and water. The organic extract was washed with brine, dried over Na₂SO₄, filtered and concentrated. The residue was subjected to silica gel chromatography eluted with 10-30% ethyl acetate in hexanes to provide methyl 2-[6-(azidomethyl)pyridin-3-yl]benzoate that gave a proton NMR spectrum consistent with theory and a mass ion (ES+) of 269.3 for M+H⁺.

To a stirred solution of methyl 2-[6-(azidomethyl)pyridin-3-yl]-benzoate (0.472 g, 1.76 mmol) in EtOH (10 mL) was added Raney 2800 nickel (slurry in water). The mixture was stirred under a H₂ atmosphere for 1 h at room temperature. The mixture was then filtered through glass filter paper, washing with additional EtOH. The resultant solution was concentrated under vacuum to provide methyl 2-[6-(aminomethyl)pyridin-3-yl]benzoate that gave proton NMR spectra consistent with theory and a mass ion (ES+) of 243.3 for M+H⁺.

A solution of 2-chloro-4-methyl-3-nitropyridine (0.342 g, 1.98 mmol) and methyl 2-[6-(aminomethyl)pyridin-3-yl]benzoate (0.436 g, 1.80 mmol) and 0.50 mL (3.6 mmol) of triethylamine (TEA) in 5 mL of THF was heated to 95° C. for 15 hours. The solvent was removed in vacuo and the crude mixture was purified using silica gel chromatography on silica gel eluted with 10-30% ethyl acetate in hexanes to provide methyl 2-(6-{[(4-methyl-3-nitropyridin-2-yl)amino]methyl}pyridin-3-yl)-benzoate that gave a proton NMR spectrum consistent with theory and a mass ion (ES+) of 379.2 for M+H⁺.

To a solution of the above material (0.263 g, 0.695 mmol) in methanol (50 mL), tin(II) chloride dihydrate (0.784 g, 3.47 mmol) was added and heated at 60° C. for 6 hours. The resulting solution was concentrated under vacuum. The residue was dissolved in ethyl acetate, and 10% aq. sodium carbonate solution was added with vigorous stirring until pH=10. The white suspension was filtered through a pad of Celite to provide crude methyl 2-(6-{[(3-amino-4-methylpyridin-2-yl)amino]methyl}pyridin-3-yl)benzoate that gave a proton NMR spectrum consistent with theory and a mass ion (ES+) of 349.2 for M+H⁺.

To a solution of the above compound (53 mg, 0.15 mmol), 3,3,3-trifluoropropionic acid, (0.025 g, 0.20 mmol), 1-ethyl-(3-dimethylaminopropyl)-carbodiimide hydrochloride (0.058 g 0.30 mmol), 1-hydroxy-7-azabenzotriazole (0.010 g, 0.15 mmol) was added triethylamine (0.054 g, 0.53 mmol). The resulting solution was stirred at room temperature for 24 hours, and partitioned between ethyl acetate and water. The organic extract was washed with brine, dried over Na₂SO₄, filtered and concentrated. The residue was subjected to silica gel chromatography eluted with 0-4% methanol in methylene chloride to provide the title compound that gave a proton NMR spectrum consistent with theory and a mass ion (ES+) of 459.3 for M+H⁺: ¹H NMR (300 MHz, MeOH-d₄) δ 8.38 (d, J=1.5 Hz, 1H), 7.93-7.90 (m, 1H), 7.80 (d, J=5.4, 1H), 7.69-7.62 (m, 2H), 7.55-4.50 (m, 1H), 7.47-7.39 (m, 2H), 6.56 (d, J=5.4 Hz, 1H), 4.86 (s, 2H), 3.66 (s, 3H), 3.47 (q, J=10.7 Hz, 2H), 2.18 (s, 3H).

The following compounds were prepared according to Method A, B of C described above using the appropriate reagents, which are either commercially available or readily prepared according to known procedures. Acid addition salts may be obtained following purification with reverse-phase HPLC using a small amount of an acid, or they may be prepared by treating the free base (FB) with the appropriate acid. The interconversion of free base to salt and vice versa is well known in the art.

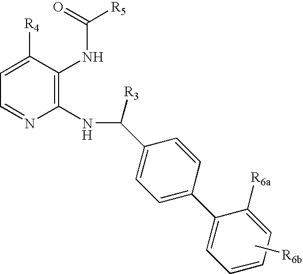

| | | | | | | | MS, | Salt |
|---|---|---|---|---|---|---|---|---|
| Ex. | R₆ₐ | R_{6b} | R₃ | R₄ | R₅ | Meth. | M⁺ + 1 | Form |
| 3 | CO₂Me | 5′-Me | Me(R) | Me | 2,2-di-F-cPr | A | 480 | HCl |
| 4 | CO₂Me | 5′-Cl | Me(R) | Me | CHF₂ | A | 474 | HCl |
| 5 | CO₂Me | 5′-Me | Me(R) | Me | CHF₂ | A | 454 | HCl |
| 6 | CN | 5′-Me | Me(R) | Me | CHF₂ | A | 421 | HCl |
| 7 | CO₂Me | H | H | H | CHF₂ | A | 412 | TFA |
| 8 | CO₂Me | H | H | Me | CF₂CF₃ | A | 494 | HCl |
| 9 | CONHMe | H | H | Me | CF₂CF₃ | A | 493 | HCl |
| 10 | CO₂Me | H | H | H | (CH₂)₂CF₃ | A | 458 | HCl |

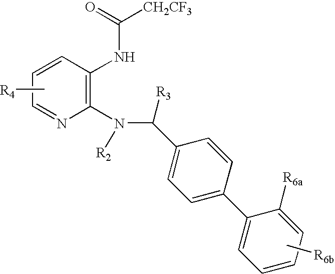

| | | | | | | | |
|---|---|---|---|---|---|---|---|
| | | | | | | MS, | Salt |
| Ex. | R₆ₐ | R_{6b} | R₃ | R₄ | Meth. | M⁺ + 1 | Form |

R2 is H unless otherwise specified

| | | | | | | | |
|---|---|---|---|---|---|---|---|
| 11 | 3-methyl-1,2,4- | 5′-F | Me(R) | 4-Cl | B | | |
| | oxadiazolyl | | | | | | |
| 12 | CONHMe | 3′-F | Me(R) | 4-Cl | B | 509 | HCl |
| 13 | 3-methyl-1,2,4- | 3′-F | H | 4-Me | A | 500 | TFA |
| | oxadiazolyl | | | | | | |
| 14 | CO₂Me | 3′-F | H | 4-Me | A | 476 | TFA |
| 15 | CO₂Me | 3′-Cl | Me(R) | 4-Me | A | 506 | FB |
| 16 | CO₂Me | 3′-Cl | H | 4-Me | A | 492 | TFA |
| 17 | CN | 3′-F | Me(R) | 4-Me | A | 457 | HCl |
| 18 | CO₂Me | 3′-F | Me(R) | 4-Me | A | 490 | TFA |
| 19 | CO₂Me | 3′-F | Me(R) | 4-Cl | B | 510 | FB |
| 20 | 5-methyl-1,2,4- | 3′-F | Me(R) | 4-Cl | B | 534 | HCl |
| | oxadiazolyl | | | | | | |
| 21 | 2-methyl-2H- | 3′-F | Me(R) | 4-Me | A | 514 | TFA |
| | tetrazol-5-yl | | | | | | |
| 22 | CO₂Me | 3′-Cl | Me(R) | 4-Cl | B | 527 | HCl |
| 23 | CO₂Me | 5′-Me | Me(R) | 4-Cl | B | 506 | HCl |
| 24 | 2-methyl-2H- | 3′-F | Me(R) | 4-Cl | B | 534 | TFA |
| | tetrazol-5-yl | | | | | | |
| 25 | CO₂Me | 3′-Cl | Me(R) | H | A | 493 | HCl |
| 26 | CONHMe | 3′-F | Me(R) | 4-Me | A | 489 | HCl |
| 27 | CO₂Me | 6′-Me | Me(R) | 4-Cl | B | 506 | HCl |
| 28 | CO₂Me | 3′-Cl | H | 4-Cl | B | 513 | FB |
| 29 | CO₂Me | 5′-F | Me(R) | 4-Cl | B | 510 | TFA |
| 30 | 5-methyl-1,2,4- | 5′-Me | H | 4-Me | A | 496 | TFA |
| | oxadiazolyl | | | | | | |
| 31 | CO₂Me | 5′-Me | Me(R) | 4-Me | A | 486 | FB |
| 32 | CO₂Me | 5′-Cl | Me(R) | 4-Cl | B | 527 | TFA |
| 33 | CF₃ | 3′-F | H | 4-Me | A | 500 | HCl |
| 34 | CO₂Me | 3′-F | Me(R) | H | A | 476 | TFA |
| 35 | CO₂Me | 6′-F | Me(R) | 4-Cl | B | 510 | TFA |
| 36 | CF₃ | 3′-F | Me(R) | 4-Cl | B | 520 | HCl |
| 37 | CO₂Me | H | Me(R) | 4-Me | A | 472 | FB |
| 38 | CO₂Me | 6′-Cl | Me(R) | 4-Cl | B | 527 | HCl |
| 39 | CONHOMe | H | Me(R) | 4-Me | A | 487 | HCl |
| 40 | SO₂NHMe | H | Me(R) | 4-Me | A | 507 | TFA |
| 41 | CONHOMe | H | H | 4-Me | A | 473 | HCl |
| 42 | CHF₂ | 3′-Cl | H | 4-Me | A | 484 | FB |
| 43 | Cl | 3′-F | Me(R) | 4-Me | A | 466 | HCl |
| 44 | CO₂Me | H | Me(R) | 4-Cl | B | 492 | HCl |
| 45 | 5-methyl-1,2,4- | H | Me(R) | 4-Cl | B | 516 | HCl |
| | oxadiazolyl | | | | | | |
| 46 | 3-methyl-1,2,4- | H | H | 4-Me | A | 482 | TFA |
| | oxadiazolyl | | | | | | |
| 47 | CN | 3′-F | H | 4-Cl | B | 463 | HCl |
| 48 | 5-methyl-1,2,4- | H | Me(R) | 4-Me | A | 496 | |
| | oxadiazolyl | | | | | | |
| 49 | 3-methyl-1,2,4- | H | H | H | A | 468 | HCl |
| | oxadiazolyl | | | | | | |
| 50 | Cl | 3′-Cl | Me(R) | 4-Me | A | 483 | HCl |
| 51 | CN | 5′-Me | Me(R) | 4-Me | A | 453 | HCl |
| 52 | Cl | 3′-F | Me(R) | 4-Cl | B | 487 | HCl |
| 53 | CF₃ | 3′-F | H | 4-Me | A | 486 | HCl |
| 54 | CF₃ | H | Me(R) | 4-Me | A | 482 | HCl |
| 55 | 5-methyl-1,2,4- | 5′-Me | Me(R) | 4-Me | A | 510 | FB |
| | oxadiazolyl | | | | | | |
| 56 | Cl | 3′-F | H | 4-Me | A | 452 | HCl |
| 57 | 1-methyl-1H- | 3′-F | Me(R) | 4-Cl | B | 534 | TFA |
| | tetrazol-5-yl | | | | | | |
| 58 | 3-methyl-1,2,4- | H | Me | 4-Me | A | 496 | HCl |
| | oxadiazolyl | | | | | | |
| 59 | CF₃ | 3′-F | Me(R) | H | A | 486 | HCl |
| 60 | CONHMe | H | Me(R) | 4-Me | A | 471 | HCl |
| 61 | 3-methyl-1,2,4- | 5′-Cl | Me(R) | 4-Cl | B | 551 | HCl |
| | oxadiazolyl | | | | | | |
| 62 | CO₂Me | H | H | 4-Me | A | 458 | HCl |
| 63 | SO₂NHMe | H | H | 4-Me | A | 493 | TFA |
| 64 | CO₂Me | H | H | 4-Cl | B | 478 | HCl |
| 65 | OCF₃ | H | Me(R) | 4-Me | A | 498 | HCl |
| 66 | 1,2,4-oxadiazol-5-yl | H | H | H | A | 454 | TFA |
| 67 | CN | H | Me(R) | 4-Me | A | 439 | HCl |
| 68 | CHF₂ | H | H | 4-Me | A | 450 | TFA |
| 69 | CONHMe | H | H | 4-Me | A | 457 | HCl |
| 70 | CO₂Me | H | H | H | A | 444 | TFA |
| 71 | Br | H | Me(R) | 4-Me | A | 493 | HCl |
| 72 | F | 3′-F | Me(R) | 4-Me | A | 450 | HCl |
| 73 | CO₂Me | 3′-Me | H | 4-Me | A | 472 | FB |
| 74 | CO₂Me | 6′-CH₂OH | H | 4-Me | A | 488 | FB |
| 75 | Cl | 3′-F | Me(R) | H | A | 453 | HCl |
| 76 | CONHOMe | 6′-Me | H | 4-Me | A | 487 | HCl |
| 77 | isoxazol-5-yl | H | H | H | A | 453 | TFA |
| 78 | Cl | 6′-Me | Me(R) | 4-Me | A | 462 | HCl |
| 79 | CO₂Me | H | Et | 4-Me | A | 486 | TFA |
| 80 | 3-methyl-1,2,4- | 6′-Me | Me(R) | 4-Me | A | 510 | TFA |
| | oxadiazolyl | | | | | | |
| 81 | 1H-1,2,4-triazol-5- | H | H | 4-Me | A | 467 | TFA |
| | yl | | | | | | |
| 82 | CO₂Me | H | H | 4-(CH₂)₂ | A | 511 | FB |
| | | | | NHCO— | | | |
| | | | | CH₂CN | | | |
| 83 | CO₂Me | 6′-CO₂Me | H | 4-Me | A | 516 | TFA |
| 84 | 4-methyl-5(4H)- | H | H | H | A | 484 | TFA |
| | oxo-1,2,4- | | | | | | |
| | oxadiazol-3-yl | | | | | | |
| 85 | CO₂Me | H | H | 4-CH₂— | A | 516 | FB |
| | | | | CO₂Me | | | |
| 86 | Cl | 5′-Cl | Me(R) | 4-Me | A | 483 | HCl |
| 87 | CO₂Me | H | H | 5-F | A | 462 | FB |
| 88 | 3-methyl-1,2,4- | 6′-Me | H | 4-Me | A | 496 | TFA |
| | oxadiazolyl | | | | | | |
| 89 | CO₂tBu | H | H | 4-Me | A | 500 | HCl |
| 90 | CO₂Me | H | H | 4-Et | A | 472 | HCl |
| 91 | F | H | Me(R) | 4-Me | A | 432 | HCl |
| 92 | 1H-pyrazol-3-yl | H | H | H | A | 452 | TFA |
| 93 | CO₂Me | H | H | 4-CH₂CN | A | 483 | FB |
| 94 | CO₂Me | 6′-CHO | H | 4-Me | A | 486 | TFA |
| 95 | CO₂Me | H | H | 5-Me | A | 458 | HCl |
| 96 | 5-(CF₃)-1,2,4- | H | H | H | A | 522 | TFA |
| | oxadiazolyl | | | | | | |
| 97 | CO₂Me | H | H | 5-Cl | A | 478 | HCl |
| 98 | CO₂Me | H | H | 4-(CH₂)₂— | A | 487 | FB |
| | | | | NH₂ | | | |
| 99 | 5(4H)-oxo-1,2,4- | H | H | H | A | 470 | TFA |
| | oxadiazol-3-yl | | | | | | |
| 100 | F | 4′-F | Me(R) | 4-Me | A | 450 | HCl |
| 101 | CO₂Me | H | H | 5-Br | A | 523 | HCl |
| 102 | 3-methyl-1,2,4- | H | H | H | A | 482 | TFA |
| | oxadiazolyl | | | | | | |
| | R2 is CH3 | | | | | | |
| 103 | CO₂Me | H | H | 6-Me | A | 458 | HCl |
| 104 | CO₂Me | H | H | 6-Cl | A | 478 | TFA |
| 105 | H | 3′-F | Me(R) | 4-Me | A | 432 | HCl |

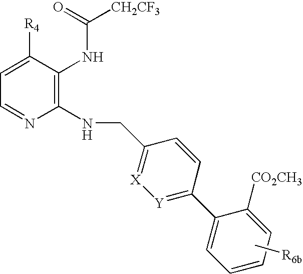

| | | | | | | MS, | Salt |
|---|---|---|---|---|---|---|---|
| Ex. | R6b | R4 | X | Y | Meth. | M⁺ + 1 | Form |
| 387 | 3′-F | Me | C(F) | CH | A | 494 | HCl |
| (1) | | | | | | | |
| 106 | 3′-F | Me | CH | C(F) | A | 494 | HCl |
| 108 | H | H | C(Me) | H | A | 458 | TFA |
| 109 | H | H | H | C(Me) | A | 458 | TFA |

What is claimed is:

1. A compound of formula I: 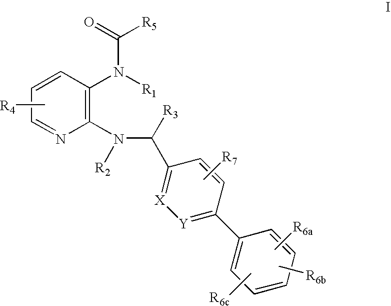
wherein
X and Y are each CH, or one is CH and the other is N;
R₁ and R₂ are independently selected from
(1) hydrogen and
(2) C₁₋₄ alkyl;
R₃ is selected from
(1) hydrogen, and
(2) C₁₋₄ alkyl optionally substituted with 1 to 4 groups selected from halogen, CO₂R^{a}, OR^{a}, COR^{a} and cyano;
R₄ is selected from
(1) hydrogen,
(2) nitro,
(3) halogen,
(4) (CH₂)ₙOR^{a},
(5) (CH₂)ₙCO₂R^{a},
(6) (CH₂)ₙCN,
(7) (CH₂)ₙNR^{b}R^{c},
(8) (CH₂)ₙNHC(O)CH₂CN,
(9) CONR^{b}R^{c}, and
(10) C₁₋₄ alkyl;
R⁵ is selected from
(1) C₃₋₆ cycloalkyl substituted with 1 or 2 fluoro,
(2) CHF₂,
(3) CH₂CF₃,
(4) CF₂CF₃, and
(5) CH₂CH₂CF₃;
R₆ₐ is selected from
(1) C₁₋₈ alkyl, optionally substituted with 1 to 5 groups independently selected from halogen, nitro, cyano, COR^{a}, SO₂R^{d}, CO₂R^{a}, NR^{b}R^{c}, NR^{b}C(O)R^{a}, NHSO₂R^{d}, OR^{a}, OC(O)R^{a}, CONR^{b}R^{c},
(2) C₃₋₈ cycloalkyl,
(3) C₂₋₈ alkenyl optionally substituted with CO₂R^{a};
(4) halogen,
(5) OCF₃,
(6) cyano,
(7) nitro,
(8) NR^{b}R^{c},
(9) NR^{b}C(O)R^{a},
(10) NR^{b}CO₂R^{a′}, wherein R^{a′} is a non-hydrogen group selected from R^{a},
(11) CO₂R^{a},
(12) COR^{a},
(13) C(O)NR^{b}R^{c},
(14) C(O)NHOR^{a},
(15) OR^{a},
(16) OC(O)R^{a},
(17) S(O)ₙR^{a′}, wherein R^{a′} is a non-hydrogen group selected from R^{a},
(18) SO₂NHR^{c},
(19) NHSO₂R^{d},
(20) C(═NOR^{a})NR^{b}R^{c},
(21) C(═NOR^{a})R^{a}, and
(22) substituted or unsubstituted heterocycle where the heterocycle is selected from oxadiazole, tetrazole, triazole, pyrazole, oxazole, isoxazole, thiazole, 4,5-dihydro-oxazole, 4,5-dihydro-1,2,4-oxadiazol-5-one, and wherein said substituent is 1 to 3 groups independently selected from C₁₋₄alkyl optionally substituted with 1 to 5 halogen atoms, OR^{a}, or OC(O)R^{a};
R_{6b} and R_{6c} are independently selected from
(1) hydrogen, and
(2) a group from R₆ₐ; with the proviso that not more than one of
R₆ₐ, R_{6b}, and R_{6c} is a heterocycle;
R₇ is selected from
(1) hydrogen,
(2) cyano,
(3) nitro,
(4) halogen,
(5) OR^{a},
(6) CO₂R^{a},
(7) CONR^{b}R^{c}, and
(8) C₁₋₄ alkyl;
R^{a} is selected from
(1) hydrogen,
(2) C₁₋₄ alkyl,
(3) C₃₋₆ cycloalkyl,
(4) aryl, and
(5) aryl-C₁₋₄ alkyl;
R^{b} and R^{c} are independently selected from
(1) hydrogen,
(2) C₁₋₄ alkyl optionally substituted with OR^{a},
(3) C₃₋₆ cycloalkyl,
(4) aryl, and
(5) aryl-C₁₋₄ alkyl; or
R^{b} and R^{c} together with the nitrogen atom to which they are attached form a 5- or 6-membered ring optionally containing a heteroatom selected from NR^{a}, O and S;
R^{d} is selected from
(1) C₁₋₄ alkyl, optionally substituted with 1 to 3 halogen atoms,
(2) aryl,
(3) aryl-C₁₋₄ alkyl, and
(4) NR^{b}R^{c};
n is 0, 1 or 2
a pharmaceutically acceptable salt thereof.

2. A compound of claim 1 wherein R₃ is hydrogen.

3. A compound of claim 1 wherein R₃ is C₁₋₄ alkyl.

4. A compound of claim 1 wherein R₄ is H or a 4-substituent.

5. A compound of claim 1 wherein R₄ is H or a 4-substituent selected from C₁₋₄ alkyl and halogen.

6. A compound of claim 1 wherein R₄ is 4-chloro or 4-methyl.

7. A compound of claim 1 wherein R⁵ is CH₂CF₃.

8. A compound of claim 1 wherein X and Y are both CH.

9. A compound of claim 1 wherein one of X and Y is CH and the other is N.

10. A compound of claim 1 wherein R₆ₐ is a 2-(or ortho-) substituent selected from CO₂R^{a}, CONR^{b}R^{c}, CONHOR^{a}, C₁₋₈ alkyl substituted with 1 to 5 halogen atoms, cyano, SO₂NHR^{c}, halogen, trifluoromethoxy, 1- and 2-methyltetrazol-5-yl, 1,2,4-oxadiazolyl, 3- and 5-methyl-1,2,4-oxadiazolyl, 3- and 5-trifluoromethyl-1,2,4-oxadiazolyl, 3-pyrazolyl, 1,2,4-triazolyl, 5-methyl-1,2,4-triazol-3-yl, and 3-methyl-1,2,4-triazol-5-yl.

11. A compound of claim 10 wherein R₆ₐ is selected from methyl carboxylate, cyano, N-methylcarboxamido and N-methylsulfonamido.

12. A compound of claim 10 wherein R₆ₐ is 1- or 2-methyltetrazolyl.

13. A compound of claim 10 wherein R₆ₐ is 1,2,4-oxadiazolyl, 5(4H)-oxo-1,2,4-oxadiazolyl, 4-methyl-5(4H)-oxo-1,2,4-oxadiazolyl, or 3- or 5-methyl-1,2,4-oxadiazolyl.

14. A compound of claim 10 wherein R₆ₐ is difluoromethyl, trifluoromethyl, trifluoromethoxy, N-methoxycarboxamide, Cl, F or Br.

15. A compound of claim 10 wherein R₆ₐ is 1,2,4-triazolyl, isoxazolyl or pyrazolyl.

16. A compound of claim 1 wherein R_{6b} is selected from hydrogen, C₁₋₈ alkyl optionally substituted with OH or 1 to 5 halogen atoms, C₂₋₆ alkenyl, NR^{b}R^{c}, OR^{a}, COR^{a}, CO₂R^{a}, NHCOR^{a}, NHSO₂R^{d} and halogen, and R_{6c} is hydrogen.

17. A compound of claim 16 wherein R_{6b} is hydrogen, halogen, CO₂R^{a}, COR^{a}, or C₁₋₄alkyl optionally substituted with hydroxy.

18. A compound of claim 16 wherein R_{6b} is hydrogen, fluoro, chloro, or methyl.

19. A compound of claim 1 represented by formula Ia: 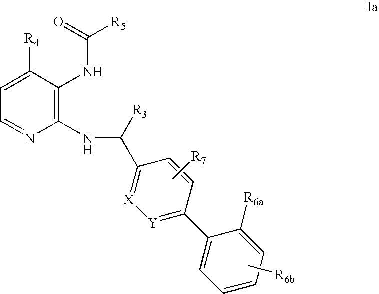
wherein R₃, R₄, R⁵, R₆ₐ, R_{6b}, R₇, X and Y are as defined in claim 1.

20. A compound of claim 19 wherein at least one of R₃, R₄ and R_{6b} is non-hydrogen.

21. A compound of claim 19 wherein at least two of R₃, R₄ and R_{6b} are non-hydrogen.

22. A compound of claim 20 wherein R₆ₐ is selected from CO₂R^{a}, CONR^{b}R^{c}, CONHOR^{a}, C₁₋₈ alkyl substituted with 1 to 5 halogen atoms, cyano, SO₂NHR^{c}, halogen, trifluoromethoxy, 1- and 2-methyltetrazol-5-yl, 1,2,4-oxadiazolyl, 3- and 5-methyl-1,2,4-oxadiazolyl, 3- and 5-trifluoromethyl-1,2,4-oxadiazolyl, 3-pyrazolyl, 1,2,4-triazolyl, 5-methyl-1,2,4-triazol-3-yl, and 3-methyl-1,2,4-triazol-5-yl.

23. A compound of claim 21 wherein R₆ₐ is selected from CO₂R^{a}, CONR^{b}R^{c}, CONHOR^{a}, C₁₋₈ alkyl substituted with 1 to 5 halogen atoms, cyano, SO₂NHR^{c}, halogen, trifluoromethoxy, 1- and 2-methyltetrazol-5-yl, 1,2,4-oxadiazolyl, 3- and 5-methyl-1,2,4-oxadiazolyl, 3- and 5-trifluoromethyl-1,2,4-oxadiazolyl, 3-pyrazolyl, 1,2,4-triazolyl, 5-methyl-1,2,4-triazol-3-yl, and 3-methyl-1,2,4-triazol-5-yl.

24. A compound of claim 19 wherein R_{6b} is hydrogen, or a 3-, 5- or 6-substituent selected from C₁₋₄alkyl and halogen.

25. A compound of claim 19 wherein
R₃ is H or C₁₋₄ alkyl;
R₄ is H, C₁₋₄ alkyl or halogen;
R⁵ is CH₂CF₃;
R₆ₐ is selected from CO₂R^{a}, CONR^{b}R^{c}, CONHOR^{a}, C₁₋₈ alkyl substituted with 1 to 5 halogen atoms, cyano, SO₂NHR^{c}, halogen, trifluoromethoxy, 1- and 2-methyl-tetrazol-5-yl, 1,2,4-oxadiazolyl, 3- and 5-methyl-1,2,4-oxadiazolyl, 3- and 5-trifluoro-methyl-1,2,4-oxadiazolyl, 3-pyrazolyl, 1,2,4-triazolyl, 5-methyl-1,2,4-triazol-3-yl, and 3-methyl-1,2,4-triazol-5-yl;
R_{6b} is selected from hydrogen, C₁₋₈ alkyl optionally substituted with OH or 1 to 5 halogen atoms, C₂₋₆ alkenyl, NR^{b}R^{c}, OR^{a}, COR^{a}, CO₂R^{a}, NHCOR^{a}, NHSO₂R^{d} and halogen; and
R_{6c} is hydrogen; with the proviso that at least one of R₃, R₄ and R_{6b} is non-hydrogen.

26. A compound of claim 26 wherein R₆ₐ is selected from CO₂R^{a}, CONR^{b}R^{c}, SO₂NHR^{c}, and cyano.

27. A compound of claim 26 wherein R₆ₐ is selected from 1- or 2-methyltetrazolyl.

28. A compound of claim 26 wherein R₆ₐ is 1,2,4-oxadiazolyl, 5(4H)-oxo-1,2,4-oxadiazolyl, 4-methyl-5(4H)-oxo-1,2,4-oxadiazolyl, or 3- or 5-methyl-1,2,4-oxadiazolyl.

29. A compound of claim 26 wherein R₆ₐ is difluoromethyl, trifluoromethyl, trifluoromethoxy, N-methoxycarboxamide, Cl, F or Br.

30. A compound of claim 26 wherein R₆ₐ is 1,2,4-triazolyl, isoxazolyl or pyrazolyl.

31. A compound of claim 1 represented by the formula Ib: 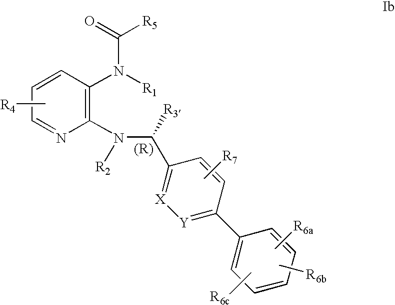
wherein all the variables are as defined in claim 1, except R_{3′} is C₁₋₄ alkyl optionally substituted with 1 to 4 groups selected from halogen, CO₂R^{a}, OR^{a}, COR^{a} and cyano.

32. A compound selected from:

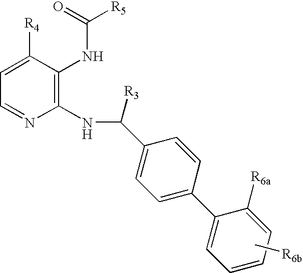

| R₆ₐ | R_{6b} | R₃ | R₄ | R₅ |
|---|---|---|---|---|
| CO₂Me | 5′-Me | Me(R) | Me | 2,2-di-F-cPr |
| CO₂Me | 5′-Cl | Me(R) | Me | CHF₂ |
| CO₂Me | 5′-Me | Me(R) | Me | CHF₂ |
| CN | 5′-Me | Me(R) | Me | CHF₂ |
| CO₂Me | H | H | H | CHF₂ |
| CO₂Me | H | H | Me | CF₂CF₃ |
| CONHMe | H | H | Me | CF₂CF₃ |
| CO₂Me | H | H | H | (CH₂)₂CF₃ |

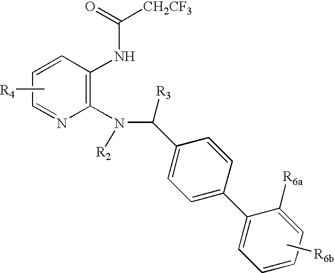
| | | | |
|---|---|---|---|
| R₆ₐ | R_{6b} | R₃ | R₄ |

R2 is H unless otherwise specified

| | | | |
|---|---|---|---|
| 3-methyl-1,2,4-oxadiazolyl | 5′-F | Me(R) | 4-Cl |
| CONHMe | 3′-F | Me(R) | 4-Cl |
| 3-methyl-1,2,4-oxadiazolyl | 3′-F | H | 4-Me |
| CO₂Me | 3′-F | H | 4-Me |
| CO₂Me | 3′-Cl | Me(R) | 4-Me |
| CO₂Me | 3′-Cl | H | 4-Me |
| CN | 3′-F | Me(R) | 4-Me |
| CO₂Me | 3′-F | Me(R) | 4-Me |
| 3-methyl-1,2,4-oxadiazolyl | 3′-F | Me(R) | 4-Cl |
| CO₂Me | 3′-F | Me(R) | 4-Cl |
| 5-methyl-1,2,4-oxadiazolyl | 3′-F | Me(R) | 4-Cl |
| 2-methyl-2H-tetrazol-5-yl | 3′-F | Me(R) | 4-Me |
| CO₂Me | 3′-Cl | Me(R) | 4-Cl |
| CO₂Me | 5′-Me | Me(R) | 4-Cl |
| 2-methyl-2H-tetrazol-5-yl | 3′-F | Me(R) | 4-Cl |
| CO₂Me | 3′-Cl | Me(R) | H |
| CONHMe | 3′-F | Me(R) | 4-Me |
| CO₂Me | 6′-Me | Me(R) | 4-Cl |
| CO₂Me | 3′-Cl | H | 4-Cl |
| CO₂Me | 5′-F | Me(R) | 4-Cl |
| 5-methyl-1,2,4-oxadiazolyl | 5′-Me | H | 4-Me |
| 3-methyl-1,2,4-oxadiazolyl | 5′-Me | H | 4-Me |
| CO₂Me | 5′-Me | Me(R) | 4-Me |
| CO₂Me | 5′-Cl | Me(R) | 4-Cl |
| CF₃ | 3′-F | H | 4-Me |
| CO₂Me | 3′-F | Me(R) | H |
| CO₂Me | 6′-F | Me(R) | 4-Cl |
| CF₃ | 3′-F | Me(R) | 4-Cl |
| CO₂Me | H | Me(R) | 4-Me |
| CO₂Me | 6′-Cl | Me(R) | 4-Cl |
| CONHOMe | H | Me(R) | 4-Me |
| SO₂NHMe | H | Me(R) | 4-Me |
| CONHOMe | H | H | 4-Me |
| CHF₂ | 3′-Cl | H | 4-Me |
| Cl | 3′-F | Me(R) | 4-Me |
| CO₂Me | H | Me(R) | 4-Cl |
| 5-methyl-1,2,4-oxadiazolyl | H | Me(R) | 4-Cl |
| 3-methyl-1,2,4-oxadiazolyl | H | H | 4-Me |
| CN | 3′-F | H | 4-Cl |
| 5-methyl-1,2,4-oxadiazolyl | H | Me(R) | 4-Me |
| 3-methyl-1,2,4-oxadiazolyl | H | H | H |
| Cl | 3′-Cl | Me(R) | 4-Me |
| CN | 5′-Me | Me(R) | 4-Me |
| Cl | 3′-F | Me(R) | 4-Cl |
| CF₃ | 3′-F | H | 4-Me |
| CF₃ | H | Me(R) | 4-Me |
| 5-methyl-1,2,4-oxadiazolyl | 5′-Me | Me(R) | 4-Me |
| Cl | 3′-F | H | 4-Me |
| 1-methyl-1H-tetrazol-5-yl | 3′-F | Me(R) | 4-Cl |
| 3-methyl-1,2,4-oxadiazolyl | H | Me | 4-Me |
| CF₃ | 3′-F | Me(R) | H |
| CONHMe | H | Me(R) | 4-Me |
| 3-methyl-1,2,4-oxadiazolyl | 5′-Cl | Me(R) | 4-Cl |
| CO₂Me | H | H | 4-Me |
| SO₂NHMe | H | H | 4-Me |
| CO₂Me | H | H | 4-Cl |
| OCF₃ | H | Me(R) | 4-Me |
| 1,2,4-oxadiazol-5-yl | H | H | H |
| CN | H | Me(R) | 4-Me |
| CHF₂ | H | H | 4-Me |
| CONHMe | H | H | 4-Me |
| CO₂Me | H | H | H |
| Br | H | Me(R) | 4-Me |
| F | 3′-F | Me(R) | 4-Me |
| CO₂Me | 3′-Me | H | 4-Me |
| CO₂Me | 6′-CH₂OH | H | 4-Me |
| Cl | 3′-F | Me(R) | H |
| CONHOMe | 6′-Me | H | 4-Me |
| isoxazol-5-yl | H | H | H |
| Cl | 6′-Me | Me(R) | 4-Me |
| CO₂Me | H | Et | 4-Me |
| 3-methyl-1,2,4-oxadiazolyl | 6′-Me | Me(R) | 4-Me |
| 1H-1,2,4-triazol-5-yl | H | H | 4-Me |
| CO₂Me | H | H | 4- |
| | | | (CH₂)₂NHCOCH₂CN |
| CO₂Me | 6′-CO₂Me | H | 4-Me |
| 4-methyl-5(4H)-oxo-1,2,4- | H | H | H |
| oxadiazol-3-yl | | | |
| CO₂Me | H | H | 4-CH₂CO₂Me |
| Cl | 5′-Cl | Me(R) | 4-Me |
| CO₂Me | H | H | 5-F |
| 3-methyl-1,2,4-oxadiazolyl | 6′-Me | H | 4-Me |
| CO₂tBu | H | H | 4-Me |
| CO₂Me | H | H | 4-Et |
| F | H | Me(R) | 4-Me |
| 1H-pyrazol-3-yl | H | H | H |
| CO₂Me | H | H | 4-CH₂CN |
| CO₂Me | 6′-CHO | H | 4-Me |
| CO₂Me | H | H | 5-Me |
| 5-(CF₃)-1,2,4-oxadiazolyl | H | H | H |
| CO₂Me | H | H | 5-Cl |
| CO₂Me | H | H | 4-(CH₂)₂NH₂ |
| 5(4H)-oxo-1,2,4- | H | H | H |
| oxadiazol-3-yl | | | |
| F | 4′-F | Me(R) | 4-Me |
| CO₂Me | H | H | 5-Br |
| 3-methyl-1,2,4- | H | H | H |
| oxadiazolyl* | | | |
| CO₂Me | H | H | 6-Me |
| CO₂Me | H | H | 6-Cl |
| H | 3′-F | Me(R) | 4-Me |

*R₂ is CH₃

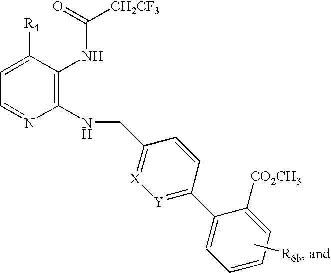

| R_{6b} | R₄ | X | Y |
|---|---|---|---|
| 3′-F | Me | C(F) | CH |
| 3′-F | Me | CH | C(F) |
| H | Me | N | CH |
| H | H | C(Me) | H |
| H | H | H | C(Me) |

33. A pharmaceutical composition comprising a compound according to claim 1 or a pharmaceutically acceptable salt thereof; and a pharmaceutically acceptable carrier.

34. A method of treatment or prevention of pain and inflammation comprising a step of administering, to a subject in need of such treatment or prevention, an effective amount of a compound according to claim 1 or a pharmaceutically acceptable salt thereof.

35. A method of treatment of osteoarthritis, repetitive motion pain, dental pain, cancer pain, myofascial pain, muscular injury pain, fibromyalgia pain, perioperative pain comprising a step of administering, to a subject in need of such treatment, an effective amount of a compound according to claim 1 or a pharmaceutically acceptable salt thereof.

36. A method of treatment or prevention of inflammatory pain caused by chronic obstructive pulmonary disease, asthma, inflammatory bowel disease, rhinitis, pancreatitis, cystitis (interstitial cystitis), uveitis, inflammatory skin disorders, rheumatoid arthritis, edema resulting from trauma associated with burns, sprains or fracture, postsurgical intervention, osteoarthritis, rheumatic disease, teno-synovitis, or gout comprising a step of administering, to a subject in need of such treatment or prevention, an effective amount of a compound according to claim 1 or a pharmaceutically acceptable salt thereof.

37. A method of treatment or prevention of pain associated with angina, menstruation or cancer comprising a step of administering, to a subject in need of such treatment or prevention, an effective amount of a compound according to claim 1 or a pharmaceutically acceptable salt thereof.

38. A method of treatment of diabetic vasculopathy, post capillary resistance, diabetic symptoms associated with insulitis, psoriasis, eczema, spasms of the gastrointestinal tract or uterus, Crohn's disease, ulcerative colitis, or pancreatitis comprising a step of administering, to a subject in need of such treatment, an effective amount of a compound according to claim 1 or a pharmaceutically acceptable salt thereof.

39. A method of treatment or prevention of pain caused by pneumoconiosis, including aluminosis, anthracosis, asbestosis, chalicosis, ptilosis, siderosis, silicosis, tabacosis, byssinosis, adult respiratory distress syndrome, bronchitis, allergic rhinitis, vasomotor rhinitis, liver disease, multiple sclerosis, atherosclerosis, Alzheimer's disease, septic shock, cerebral edema, headache, migraine, closed head trauma, irritable bowel syndrome, or nephritis comprising a step of administering, to a subject in need of such treatment or prevention of pain, an effective amount of a compound according to claim 1 or a pharmaceutically acceptable salt thereof.
